(19) United States
(12) Patent Application Publication
(10) Pub. No.: US 2002/0077491 A1
(43) Pub. Date: Jun. 20, 2002

(54) Methods for forming combinatorial libraries combining amide bond formation with epoxide opening

(76) Inventors: Gerald W. Shipps, Stoneham, MA (US); Kristin E. Rosner, Cambridge, MA (US); Gergely M. Makara, Quincy, MA (US); Edward A. Wintner, Cambridge, MA (US); Huw M. Nash, Cambridge, MA (US); Jason S. Felsch, Waltham, MA (US); Kollol Pal, Needham, MA (US); George R. Lenz, Andover, MA (US)

Correspondence Address: Janice M. Klunder, Hale and Dorr, 60 State Street, Boston, MA, 02109, US

(21) Appl. No.: 09/943,852

(22) Filed: Aug. 31, 2001

Related U.S. Application Data
(60) Provisional application No. 60/230,122, filed on Sep. 5, 2000.

(51) Int. Cl. G01N 33/53; C07C 269/00; G01N 33/543; C07D 303/00

(52) U.S. Cl. 549/513; 435/7.1; 436/518; 560/24; 564/32; 564/80; 564/143

(57) ABSTRACT

The invention relates to methods for forming combinatorial libraries. The invention provides methods suitable for the rapid and convenient synthesis of very large combinatorial libraries of small organic molecules. In particular, the invention provides a method for forming combinatorial libraries combining amide bond formation with epoxide opening.

### BACKGROUND OF THE INVENTION

1. Field of the Invention

This invention relates to methods for forming combinatorial libraries.

2. Background of the Invention

Pharmaceutical research depends on the identification of compounds that are capable of modulating biological reactions. One approach to finding these compounds is through rational drug design. In rational drug design, the structure of a target molecule (e.g., a protein) is determined, and potential ligands for the target are designed based on this structural information. Alternatively, compounds which are structurally related to known ligands of the target are prepared and tested for biological activity. Ideally, the structures of both the target and ligands are known, and new ligands are designed to optimize the structural complementarity of the target and ligand.

In many cases, however, lead compounds are best discovered by random screening approaches. This is especially true for those targets whose structures are unknown and for which there are no known ligands. For screening approaches to be successful, large numbers of structurally diverse compounds must be tested to increase the probability that one or more of the tested compounds will exhibit the desired activity. As screening technology has improved, a shortage in available compounds for testing has emerged as a limiting factor in drug discovery.

To address this demand, chemists have developed methodologies for the rapid synthesis of large combinatorial libraries of peptides, oligonucleotides, and small organic molecules. In combinatorial chemistry, a large number of compounds are produced, either in the same reaction vessel, or in separate vessels. The entire combinatorial library is then assayed, and active molecules are identified, isolated if necessary, and analyzed.

Due to the poor pharmacokinetic properties of many peptides and oligonucleotides, combinatorial chemistry efforts have increasingly focused on libraries of small organic molecules, as further described in Rebek et al., U.S. Pat. No. 5,877,030, and in Lenz, “Optimizing Small Molecule Drug Targets: Focus on Combinatorial Chemistry,” Decision Resources, Mar. 31, 1998, the entire contents of which are herein incorporated by reference. However, only a limited number of synthetic methods have been adapted for the preparation of large libraries of small organic molecules. There is thus a need in the art for new methods for the rapid and convenient synthesis of very large combinatorial libraries.

### BRIEF SUMMARY OF THE INVENTION

The present invention provides methods suitable for the rapid and convenient synthesis of very large combinatorial libraries of small organic molecules. In particular, the method of the invention comprises contacting a core molecule having at least two different reactive functional groups with a mixture of nucleophilic building blocks to form a set of compounds. Thus, according to the method of the invention, a library containing a wide variety of difunctionalized compounds can be easily prepared in a one-pot reaction.

In a first aspect, the invention provides a method of forming a combinatorial library of compounds, the method comprising reacting a plurality of core molecules with a mixture of nucleophilic building blocks in a reaction vessel to form a library of compounds, wherein each of the core molecules comprises (i) an acid halide, sulfonyl halide, isocyanate or isocyanate equivalent, or activated ester functional group; and (ii) an epoxide functional group.

In a preferred embodiment according to this aspect of the invention, the core molecule has the formula

A-B-C,

wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an epoxide functional group.

In some preferred embodiments, A has the formula

—Y¹—W,

where:

W is an isocyanate or isocyanate equivalent, acid halide, or sulfonyl halide functional group, or W has the formula

—C(O)—OR¹,

where R¹ is selected from the group consisting of imido, haloalkyl, and aryl substituted with at least one electron withdrawing substituent;

Y¹ is absent or comprises a linking chain of from 1 to about 6 contiguous atoms independently selected from the group consisting of carbon, nitrogen, oxygen, or sulfur, wherein the carbon and nitrogen atoms may be optionally substituted and the nitrogen and sulfur atoms may be optionally oxidized, and wherein any of the contiguous atoms of the chemical linkage may form part of a ring structure; and

C has the formula

—Y²—Z,

where Z is an epoxide, which may be optionally substituted with an alkyl, aryl, aralkyl, or carboalkoxy group, and Y² is as defined above for Y¹.

In a second aspect, the invention provides a combinatorial library of compounds, wherein each of said compounds is produced from the reaction of a core molecule, having (i) an acid halide, sulfonyl halide, isocyanate or isocyanate equivalent, or activated ester functional group; and (ii) an epoxide functional group, with a mixture of nucleophilic building blocks.

In a third aspect, the invention provides a compound of the formula

A-B-C,

wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an epoxide functional group.

### BRIEF DESCRIPTION OF THE FIGURES

FIG. 1 is an overview of the creation and analysis of a combinatorial library.

FIG. 2 is a depiction of pharmacologically active core ring systems. Below each structure is the approximate number of drugs in the Compendium of Medicinal Chemistry that contain that structure.

FIG. 3 is a depiction of pharmaceutical agents that comprise a β-hydroxyamine moiety.

FIG. 4 is a schematic representation of a preferred method for screening the libraries of the invention.

FIG. 5 shows an example set of compatible amine building blocks for use in the method of the invention.

FIG. 6 shows the valving arrangement used in ALIS screening.

FIG. 7 shows mass spectral analysis of an affinity screening experiment with DHFRec. (a) Extracted ion chromatogram of m/z 515.2 (M+H)⁺ from ALIS experiment with DHFRec and the library of Example 15. (b) XIC of m/z=515.2 from control experiment (no library). (c) Mass spectrum of ligand 29 from (a). (d) LC-MS-MS spectrum of the early-eluting isomer 29. (e) LC-MS-MS spectrum of the late-eluting isomer 30. (f) MS-MS spectrum of 29 from an ALIS experiment with DHFRec and the sublibrary of Example 17. (g) Diagnostic fragmentation pattern used for isomer assignment of 30.

FIG. 8 shows the results of competitive ALIS experiments with (S)-29 and known DHFRec inhibitors.

FIG. 9 shows that (S)-29 exhibits specificity for E. coli DHFR over bovine (B. taurus) DHFR.

### DETAILED DESCRIPTION OF THE PREFERRED EMBODIMENTS

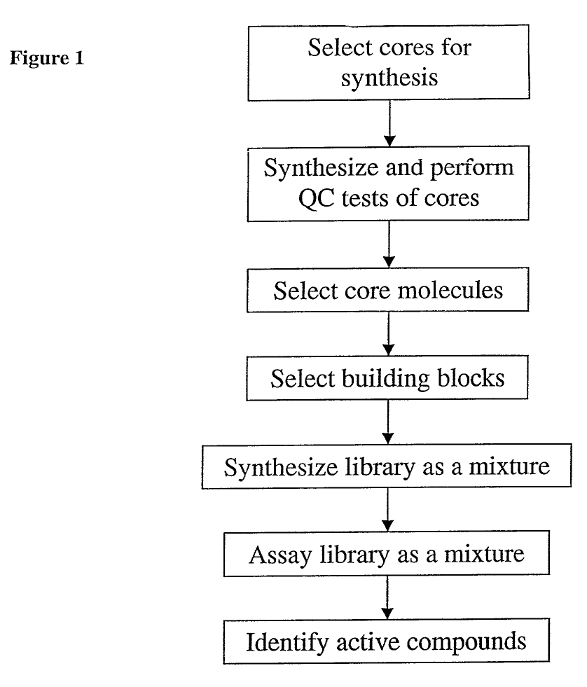

The present invention provides methods suitable for the rapid and convenient synthesis of very large combinatorial libraries of small organic molecules. In particular, the invention provides a method for forming combinatorial libraries combining amide bond formation with epoxide opening. A schematic representation of the method of the present invention is shown in FIG. 1.

The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

In a first aspect, the invention provides a method of forming a combinatorial library of compounds, the method comprising reacting a plurality of core molecules with a mixture of nucleophilic building blocks in a reaction vessel to form a library of compounds, wherein each of the core molecules comprises (i) an acid halide, sulfonyl halide, isocyanate or isocyanate equivalent, or activated ester functional group; and (ii) an epoxide functional group. According to the invention, a large number of structurally diverse compounds are conveniently synthesized in a single reaction vessel. Moreover, the library compounds produced by the present method have greater structural complexity than those prepared by other available one-pot methods for preparation of combinatorial libraries, because the core molecules used in the present method comprise at least two different reactive functional groups.

In particular, in preferred embodiments, the libraries produced by the present ;)O method comprise compounds having both β-hydroxyamine functional groups and amide, sulfonamide, or urea functional groups. In preferred embodiments, at least 90%, 95%, or 99% of the library compounds each comprise a β-hydroxyamine functional group and an amide, sulfonamide, or urea functional group.

The core molecule serves as a scaffold to which building blocks can be linked. In some embodiments, the core molecule is rigid. In the library compounds produced from such rigid core molecules, the relative position of building block moieties is fixed. In some other embodiments, the core molecule comprises a rigid portion and a non-rigid portion. In library compounds produced from these core molecules, rotation about rotatable bonds in the non-rigid portion allows for variation in the position of building blocks relative to each other and to the rigid portion of the molecule. The dynamic variability of these compounds permits conformational changes in situ, which can enhance the probability of binding to a given target.

In some embodiments, the core molecule is a dicore, and has just one acid halide, sulfonyl halide, isocyanate or isocyanate equivalent, or activated ester functional group and one epoxide functional group. In some other embodiments, the core molecule is a tricore, and has three reactive functional groups, preferably one epoxide functional group and two acid halide, sulfonyl halide, isocyanate or isocyanate equivalent, or activated ester functional groups. In yet other embodiments, the core molecule is a tetracore having four reactive functional groups, preferably one epoxide functional group and three acid halide, sulfonyl halide, isocyanate or isocyanate equivalent, or activated ester functional groups.

In a preferred embodiment according to this aspect of the invention, the core molecule has the formula

A-B-C,

wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an epoxide functional group.

In some preferred embodiments, B comprises at least one aromatic ring. In some preferred embodiments, B comprises at least one heterocyclic ring. In some preferred embodiments, the heterocyclic ring is aromatic. In some preferred embodiments, B comprises a fused bicyclic or tricyclic ring system. In some other preferred embodiments, B comprises two rings connected by a covalent bond.

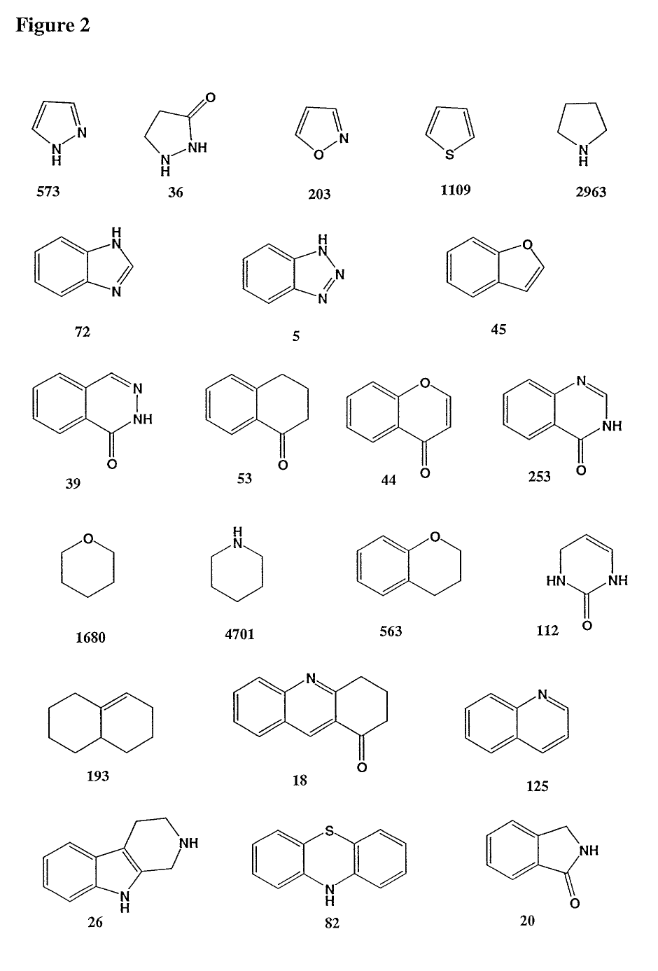

In certain preferred embodiments, B comprises a ring system with known pharmacological activity. Non-limiting examples of such pharmacologically active ring systems contemplated within the scope of the invention are depicted in FIG. 2. Below each structure is the approximate number of drugs in the Compendium of Medicinal Chemistry (CMC-3d Release 94.1; MDL Information Systems, Inc., San Leandro, Calif.) that contain that structure. The present invention provides methods for rapidly synthesizing large numbers of structural analogs of these drugs. The libraries produced by the method of the invention and the component library compounds are thus also expected to exhibit biological activity.

It will be recognized by one skilled in the art that the groups A and C may be attached to B. including the ring systems shown in FIG. 2, at any ring positions that result in a stable compound. Furthermore, A and C may be attached to the same or to different rings. The choice of position of attachment for the groups A and C will be guided by such factors as ease of synthesis and desired proximity of the building block moieties in the resultant library compounds. If A and C are attached at adjacent positions of a rigid ring, then the building block moieties will be positioned relatively close to each other in the library compounds. If A and C are attached at opposite positions of a rigid ring, then the building block moieties will be held relatively far apart in the library compounds.

In some preferred embodiments, A has the formula

—Y¹—W,

where:

W is an isocyanate or isocyanate equivalent, acid halide, or sulfonyl halide functional group, or W has the formula

—C(O)—OR¹,

where R¹ is selected from the group consisting of imido, haloalkyl, and aryl substituted with at least one electron withdrawing substituent;

Y¹ is absent or comprises a linking chain of from 1 to about 6 contiguous atoms independently selected from the group consisting of carbon, nitrogen, oxygen, or sulfur, wherein the carbon and nitrogen atoms may be optionally substituted and the nitrogen and sulfur atoms may be optionally oxidized, and wherein any of the contiguous atoms of the chemical linkage may form part of a ring structure.

Preferably, the acid halide or sulfonyl halide is an acid chloride or sulfonyl chloride.

In some preferred embodiments, W is —C(O)—OR¹, where R¹ is selected from the group consisting of imido, haloalkyl, and aryl substituted with at least one electron withdrawing substituent. In embodiments wherein R¹ is imido, the imido group is preferably attached via its nitrogen atom, with —OR¹ being a radical derived from the corresponding N-hydroxyimide, preferably N-hydroxysuccinimide or N-hydroxyphthalimide.

In embodiments wherein R¹ is haloalkyl, it is preferably perhaloalkyl, more preferably perfluoroalkyl, including, without limitation, trifluoromethyl, pentafluoroethyl, or heptafluoropropyl.

When R¹ is aryl, it is substituted with an electron withdrawing substituent preferably selected from the group consisting of chloro, fluoro, and nitro. More preferably, R¹ is selected from the group consisting of pentafluorophenyl, dinitrophenyl, nitrophenyl, difluorophenyl, fluorophenyl, trifluorophenyl, chlorophenyl, dichlorophenyl, chloronitrophenyl, and tetrafluoronitrophenyl.

In certain preferred embodiments, Y¹ is absent, and the acid halide, sulfonyl halide, isocyanate or isocyanate equivalent, or activated ester functional group is covalently attached directly to a ring in B. In certain other preferred embodiments, Y¹ is C₁-C₆ alkylene or C₂-C₆ alkenylene, preferably C₁-C₄ alkylene or C₂-C₄ alkenylene, any which groups may be optionally substituted. In some preferred embodiments, the Y¹ linking chain comprises an ester, amide or sulfonamide linkage. In some other preferred embodiments, the Y¹ linking chain comprises an ether linkage.

In some preferred embodiments, the epoxide ring in C is fused to a ring in B. In some other preferred embodiments, C has the formula

—Y²—Z,

where Z is an epoxide, which may be optionally substituted with an alkyl, aryl, aralkyl, or carboalkoxy group, and Y² is as defined above for Y¹.

In certain preferred embodiments, Y² is absent, and the epoxide ring is covalently attached directly to a ring in B. In certain other preferred embodiments, Y² comprises a ring, and Z is a spiroepoxide attached to the ring. In yet other preferred embodiments, Y² is C₁-C₆ alkylene or C₂-C₆ alkenylene, preferably C₁-C₄ alkylene or C₂-C₄ alkenylene, any of which groups may be optionally substituted. In yet other preferred embodiments, the Y² linking chain comprises an ester, amide or sulfonamide linkage. In still yet other preferred embodiments, the Y² linking chain comprises an ether linkage.

In some preferred embodiments, A comprises two W groups or C comprises an epoxide and a W group. Preferably, A has the formula

—Y¹—W,

as defined above, where Y¹ comprises a W group attached to the linking chain, or C has the formula

—Y²—Z,

as defined above, where Y² comprises a W group attached to the linking chain.

The epoxide functional group of the core molecule is typically introduced by epoxidation of a core precursor; by manipulation of vicinal diol groups on a core precursor, e.g., internal Mitsunobu-type reactions or acylation or sulfonylation of one alcohol followed by displacement by the adjacent alcohol; by alkylation of a core precursor with an epoxide-containing moiety, including, without limitation, an epoxy halide or an epoxy sulfonate; by Mitsunobu reaction of a core precursor with an epoxyalcohol; by alkylation of a core precursor with a group that can be readily converted to an epoxide, including, without limitation, allyl bromide or 2,3-dibromo-1-propanol; or by carbene/carbenoid insertions into ketones or aldehydes.

Synthetic routes for the preparation of a variety of core molecules within the scope of the present invention are illustrated in Equations 1-42.

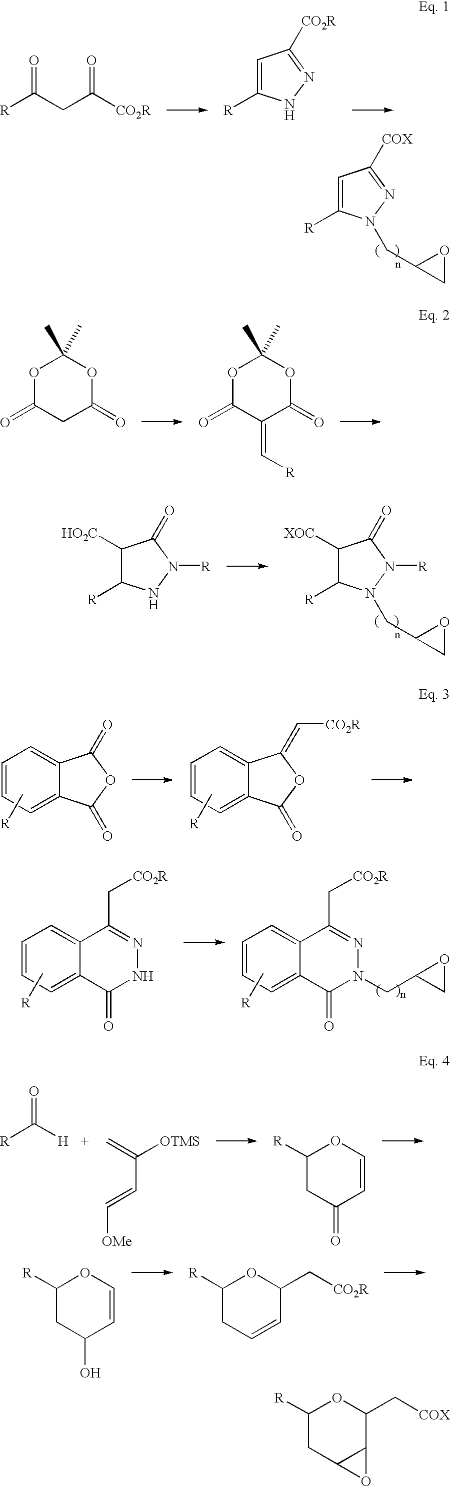

One of skill in the art would recognize that a number of reaction conditions may be utilized to carry out the transformations illustrated in these reaction sequences and that other routes may also be suitable for synthesis of the same molecules. Furthermore, one of skill in the art would recognize that other core molecules within the scope of the present invention may be synthesized by adaptation of the illustrated routes or known procedures. The synthesis of core molecules is further described in the Examples.

The core molecules are contacted with a mixture of nucleophilic building blocks to form a library of compounds. Building blocks have at least one functional group that reacts with the reactive centers of the cores to form the combinatorial library molecules. The building blocks can include more than one functional group, e.g., a first functional group for reacting with a core and a second functional group that can be protected with a protecting group. The protecting group can be removed under conditions different than those for the reaction of the core with the building blocks. The protecting groups are reversibly attached to the functional groups and can be removed from the functional groups and/or library molecules. Standard functional group protecting groups are well known to those of skill in the art and may be found described in Greene and Wuts, Protective Groups in Organic Synthesis, 2^{nd} Edition, John Wiley & Sons: New York, 1991, and in Kocienski, Protecting Groups, Georg Thieme Verlag: New York, 1994.

In some preferred embodiments, the nucleophilic building blocks are amines, preferably primary or secondary amines. The amines may be arylamines, including anilines, heteroarylamines, or alkylamines. Other nonlimiting examples of nucleophilic building blocks suitable for use in the present invention include thiols and alcohols, either of which may be either alkyl, aryl, or heteroaryl.

In some embodiments, the building blocks are preferably chosen so that each building block can react with each of the reactive centers on the core molecule. For example, amines can react with activated ester groups, with acid halide groups, and with epoxides.

In one embodiment, building blocks are chosen to provide for maximum diversity of the library that results when the building blocks and the cores are combined. This is preferably done by first grouping building blocks into sets of building blocks having similar molecular shape and functionality. For example, one such group of building blocks may be characterized in that all member building blocks have a non-aromatic ring and a tertiary amine. A set of building blocks to be reacted with a core molecule is then chosen such that the set comprises at least one building block from each group.

In another embodiment, building blocks are chosen to provide for a high degree of similarity between the library compounds. The method according to this embodiment of the invention is particularly useful when optimizing for a particular activity once a lead compound has been identified.

The criteria used to select the individual building blocks for inclusion into sets of building blocks for reaction with the core molecules are further elaborated below.

In one embodiment, the building blocks are selected such that each of the compounds in the resulting library has a different molecular weight than all of the other compounds in the library. The library produced according to this embodiment is referred to as a “mass-coded library”. The percentage of compounds having a molecular weight that is the same as the molecular weight of another compound in the library is referred to as the “mass redundancy” of the library.

The selection of building blocks is done, for example, as described in Nash et al., WO 99/35109, which is hereby incorporated by reference in its entirety. Briefly, a set of building blocks that can react with a core molecule having n reactive functional groups is selected. The set is preferably selected such that at least about 80%, 85%, 90% or 95% of the possible combinations of n building blocks derived from the set have an exact molecular mass sum that, at a resolution of 2 decimal places, is distinct from the molecular mass sum of any other combination of n building blocks derived from the set. The molecular mass sum of a combination of building block moieties is the sum of the masses of each building block moiety within the combination. Since the core is the same for each compound in the library, the differences in the molecular masses of the library compounds depend on the building blocks, and not the core. For the purposes of the invention, two molecular masses are distinct if they can be distinguished by mass spectrometry or high resolution mass spectrometry. For example, molecular masses that differ by at least 0.05 atomic mass units (AMU) can be distinguished by high resolution mass spectrometry.

In another embodiment, nucleophilic building blocks are first subjected to a test protocol to determine suitability for library synthesis. Potential building blocks are first contacted with a test core molecule having only one reactive functional group. For example, activated esters readily condense with a wide variety of amines in high yield. Therefore, a test core molecule having only an epoxide functional group is preferably contacted with potential amine building blocks. To pass the first stage of the test protocol, a potential building block must react with the test core to give a product with a mass spectrum consistent with the predicted product, and the product preferably must be >75% pure, more preferably >80% pure, and still more preferably >85% pure as determined by a standard analytical method such as high performance liquid chromatography (HPLC).

In general, both primary and secondary amines react with epoxides at satisfactory rates. However, the product of epoxide opening with an unbranched primary amine may further react to afford unwanted oligomeric compounds. Therefore, in some preferred embodiments, unbranched primary amines are preferably excluded from the pool of potential building blocks. The pool of potential building blocks thus preferably comprises a highly diverse initial set of secondary amines and branched primary amines. Structural and functional analogues of all potential building blocks are preferably subjected to the test protocol.

After confirming that representative individual nucleophilic building blocks each react with the test core to form a pure compound, other experiments are conducted to show that a set of representative building blocks reacts with a core molecule having at least two reactive centers to form a high percentage of the possible combinations. In other words, building blocks must be competitive in reactivity in order to be included in the final set of compatible amines. Preferably, when a set of amine building blocks is contacted with a di- or tricore, >60% (dicore) or >70% (tricore) of all possible combinations are easily detected by mass spectrometry (MS) or liquid chromatography/mass spectrometry (LC/MS).

By this procedure, it is possible to maximize the number of distinct library compounds that will be produced by contacting the core molecule with the set of amine building blocks. However, it is not possible by this procedure to determine whether all positional isomers are represented in the library. Positional isomers are library compounds that each have the same combination of building blocks, but where the building blocks are present at different positions of the core molecule. Because positional isomers have identical masses, the mass spectrometer is not able to differentiate between them. However, they can be distinguished using other analytical tools, including MS/MS analysis.

In some particularly preferred embodiments, potential building blocks are first subjected to the test protocol described above to select a set of compatible building blocks. From the set of compatible building blocks, a final set of building blocks is then selected such that each of the compounds in the resulting library has a different molecular weight than all of the other compounds in the library. Preferably, the set is selected such that at least about 80%, 85%, 90% or 95% of the possible combinations of n building blocks derived from the set have an exact molecular mass sum that, at a resolution of 2 decimal places, is distinct from the molecular mass sum of any other combination of n building blocks derived from the set.

To form a combinatorial library of compounds, a plurality of core molecules, as described above, is contacted with a mixture of nucleophilic building blocks, as described above. Preferably, the core molecules are contacted with the mixture of nucleophilic building blocks in a single reaction vessel. In some embodiments, a plurality of identical core molecules is contacted with the mixture of nucleophilic building blocks. In some other preferred embodiments, the plurality of core molecules comprises two or more different core molecules. When multiple different core molecules are contacted with the mixture of nucleophilic building blocks in a single reaction vessel, the core molecules must be compatible. That is, each core molecule must be capable of reacting with each nucleophilic building block, with essentially no reaction occurring between core molecules.

In some preferred embodiments, the reaction vessel also contains a solvent, preferably a solvent in which the core and building blocks are soluble. Nonlimiting examples of useful solvents include tetrahydrofuran (THF), dichloromethane (DCM), toluene, isopropanol, dimethylsulfoxide (DMSO), dimethyl formamide (DMF), dimethyl ether (DME), ethanol, and mixtures of these solvents. Some preferred solvent mixtures include THF-DCM and THF-DCM-isopropanol, wherein THF preferably constitutes up to 10% of the solvent mixture. Preferably, in solvent mixtures comprising DCM and isopropanol, the two solvents are present in a 2:1 ratio. In some particularly preferred embodiments, a 2:1 DCM-isopropanol solvent mixture containing a trace amount of THF is used.

In some preferred embodiments, base is added to the reaction mixture to accelerate the addition of building blocks to the reactive centers on the cores. Useful bases include tertiary or aromatic amine bases, including, without limitation, triethylamine (Et₃N, TEA), diisopropylethylamine (DIEA) and pyridine. In certain embodiments, DIEA is the preferred base since TEA may form adducts with the core molecule. No DIEA-core adducts have been detected by MS or HPLC.

The building blocks are contacted with the core molecules under conditions and for a time sufficient for the reaction of each reactive center of the core molecule with a building block. In certain preferred embodiments, the reactions at acid halide, sulfonyl halide, isocyanate or isocyanate equivalent, or activated ester sites and the reactions at epoxide sites are performed sequentially to prevent esterification or oligomerization of the amino alcohol produced upon epoxide opening. In this embodiment, the core molecules are first incubated with the building blocks and a base for a time sufficient to promote amide formation between the amine building blocks and the acid halide or activated ester sites on the core molecule. A period of 1-24 hours, preferably 1-10 hours, and more preferably 1-5 hours, is generally sufficient to achieve complete reaction of the acid halide and activated ester sites. Only a minimal amount of epoxide opening is generally observed in this time period.

To promote reaction of the amine building blocks with the epoxide centers of the core molecule, a Lewis acid may be added to the reaction mixture. Lewis acid catalysts suitable for catalyzing epoxide ring opening include, without limitation, lanthanide (III) salts, preferably triflates, transition metal salts or complexes, and lithium perchlorate. Preferably, the Lewis acid catalyst is selected from the group consisting of lanthanide(III) triflates, scandium(III) salts, yttrium(III) salts, copper(II) triflate, cobalt(II) chloride, tin(II) triflate, titanium(IV) isopropoxide, and lithium perchlorate. In some embodiments, the Lewis acid catalyst may be bonded to, or encapsulated by, a polymeric resin or material. Preferably, the Lewis acid catalyst is added in an amount from about 5 mol % to about 25 mol %, more preferably from about 10 mol % to about 20 mol %, and most preferably about 15 mol %. In some preferred embodiments, the Lewis acid may be added in more than one portion. Preferably, additional base, preferably a tertiary or aromatic amine base, is also added. Polymeric basic resins appear to be incompatible with the Lewis acid due to strong complexation and catalyst deactivation.

For some core molecules, heating of the reaction mixture may be required to ensure complete reaction of the epoxide functional groups. In some preferred embodiments, the reaction mixture is conveniently heated in a sealed tube. Preferably, the reaction mixture is heated at a temperature from about 35° C. to about 120° C., more preferably from about 35° C. to about 65° C., and still more preferably from about 45° C. to about 50° C.

In certain particularly preferred embodiments, the reaction mixture is stirred for a total of about 30 hours at 45-50° C. In general, the order of epoxide reactivity under these library-generating conditions (see below) is consistent with generally observed epoxide opening results.

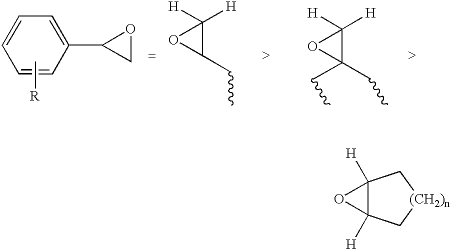

Although different epoxides react at different rates, no deleterious effects are generally observed when the reactions are conducted at elevated (45-50° C.) temperatures. Therefore, libraries are preferably generated at elevated temperature to facilitate epoxide opening. Finally, it is well established that reactions become less selective as temperature is increased, a desirable effect in the method of the present invention, because it minimizes reactivity differences between amine building blocks.

In embodiments employing a Lewis acid catalyst, the catalyst is preferably removed from the product library to prevent possible Lewis acid catalyzed degradation of library compounds. Polymeric resins exhibit high affinity for Lewis acids and may be conveniently used to deplete remaining Lewis acid catalyst from the library solution. This was demonstrated visually by using a known Yb(III) complexation reagent, glyoxal bis(2-hydroxyanil), which forms an intensely red coordination compound [Feigl, F.; Anger, V. Spot tests in Inorganic Analysis; Elsevier: New York, 1972]. Dried amberlite IRA-67 was added to reaction mixtures containing Yb(III) salts, and the mixtures were filtered after one hour. To the filtrate was added glyoxal bis(2-hydroxyanil) and 1,8-diazabicyclo[5.4.0]undec-7-ene (DBU), resulting in a pink solution. This result was in stark contrast to a control reaction lacking amberlite, which was an intense blood-red color. Aminomethylpolystyrene had a similar depletion effect.

In preferred embodiments, the amounts of core molecules and building blocks used in the coupling reaction are selected so that the molar ratio of building block functional groups to core reactive centers is equal to, or slightly greater than, one. This selection criterion enhances the likelihood that each core reactive center will be linked to a building block and that each building block (regardless of whether it contains a strong or a weak nucleophilic functional group) will react with a core reactive center. For example, 10 moles of a core having 4 reactive centers is optimally contacted with a total of 40 moles, or slightly more than 40 moles, of a plurality of amine building blocks.

If the molar ratio of building block functional groups to reactive centers is significantly greater than 1.0, the diversity of the combinatorial library may be limited, because only the most reactive functional groups will react with the reactive centers. If the molar ratio of building block functional groups to reactive centers is less than 1.0, the combinatorial library compounds may include unreacted reactive centers. Preferably, a ratio of slightly greater than one (e.g., 1.1 building block functional groups per core reactive site) is employed to maximize total reactivity with the cores. Because epoxides react at a slower rate than do acid halides or activated esters, a portionwise addition of amines may preferably be implemented so that the effective ratio of 1:1 is maintained throughout the reaction.

Using the techniques described above, libraries can be generated in which each member of the library is present in the library at approximately the same concentration (preferably within 100%, more preferably within 20%, and still more preferably within 10%) as any other member of the library. In determining the concentration of each member of the library, however, the apparent concentration must be normalized by a factor that takes into account the redundancy of positional isomers. As stated above, the mass spectrometer cannot distinguish between positional isomers, and all positional isomers must be considered to be the same compound. For example, for a library made with a core having three reactive centers and building blocks A and B, the compounds AAB, ABA, and BAA are indistinguishable. Therefore, assuming a statistical distribution of building blocks, the apparent concentration of an AAB molecule will be three times greater than the concentration of an AAA molecule.

In a preferred embodiment, the library contains at least about 100 different library compounds, more preferably at least 500, 1000, 5000, or more different library compounds. Preferably, each library compound is present in the library at the same approximate concentration as any other member of the library.

In a second aspect, the invention provides a combinatorial library of compounds, wherein each of said compounds is produced from the reaction of a core molecule, having (i) an acid halide, sulfonyl halide, isocyanate or isocyanate equivalent, or activated ester functional group; and (ii) an epoxide functional group, with a mixture of nucleophilic building blocks.

In preferred embodiments, at least one nucleophilic building block is an amine.

Some preferred libraries produced by the present method comprise compounds having both β-hydroxyamine functional groups and amide, sulfonamide, or urea functional groups. In preferred embodiments, at least 90%, 95%, or 99% of the library compounds each comprise a β-hydroxyamine functional group and an amide, sulfonamide, or urea functional group. If the core molecule has more than one acid halide, sulfonyl halide, isocyanate or isocyanate equivalent, or activated ester functional group, then a library compound may have more than one amide, sulfonamide, or urea functional group. If the core molecule has more than one epoxide functional group, then a library compound may have more than one β-hydroxyamine functional group. The β-hydroxyamine and amide, sulfonamide, or urea functional groups have electronic properties that make them useful for binding to biomolecules, including, without limitation, enzyme active sites, receptor binding sites, and other protein pockets. For example, the β-hydroxyamine and amide, sulfonamide, or urea groups can participate in hydrogen bonding with protein residues.

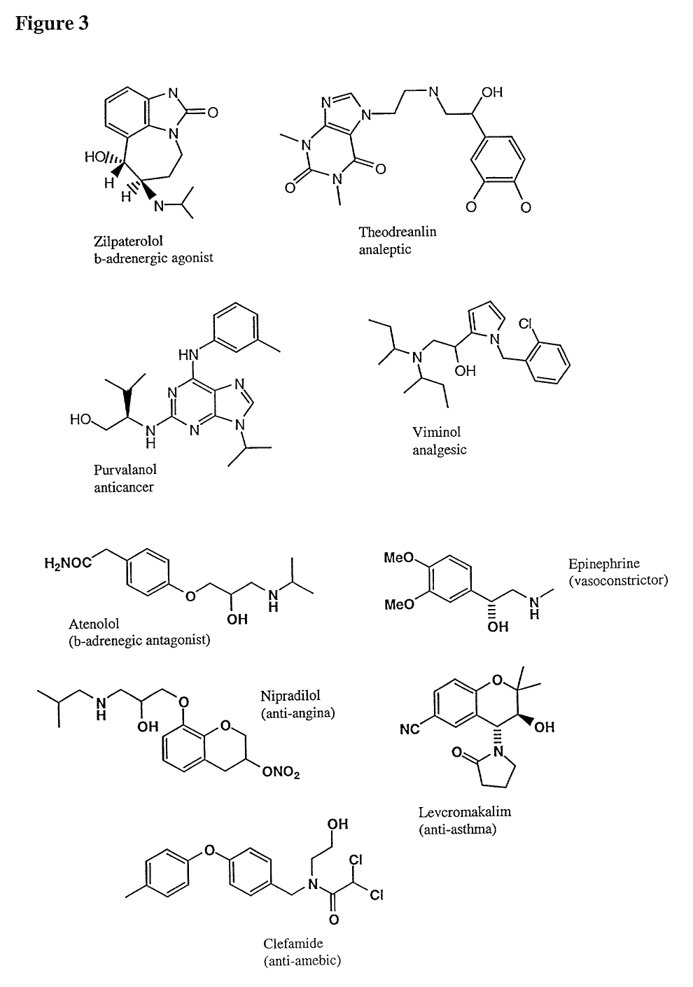

The β-hydroxyamine moiety is found in a number of pharmaceutical agents, with activity as analeptics, analgesics, β-adrenergic agonists, β-adrenergic antagonists, and anticancer agents. Examples of such useful pharmaceutical agents are shown in FIG. 3. Because the library compounds produced by the method of the present invention also contain the β-hydroxyamine pharmacophore, they may be expected to exhibit similar useful biological activities. Thus, in certain preferred embodiments, the libraries of the invention are useful as β-adrenergic agonists or antagonists. Preferably, the libraries comprise compounds with β-adrenergic agonist or antagonist activity. In certain other preferred embodiments, the libraries of the invention possess analeptic, analgesic or anticancer activity, or comprise compounds with such activity.

In some other preferred embodiments, the libraries produced by the method of the present invention possess biological activity selected from the group consisting of protease inhibitory activity, kinase inhibitory activity, antibacterial activity, antifungal activity, and anticancer activity. In certain particularly preferred embodiments, the library produced by the method of the present invention is useful as an antagonist of E. coli dihydrofolate reductase (DHFRec), a known target for antibacterial, antiprotazoal, and anticancer drug therapy. The library preferably comprises compounds with DHFRec inhibitory activity.

In some preferred embodiments according to this aspect of the invention, the core molecule has the formula

A-B-C,

wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an epoxide functional group.

Preferred values for A, B, and C are as described above for the first aspect of the invention.

In certain preferred embodiments, B comprises a ring system with known pharmacological activity. In these embodiments, the libraries produced by the method of the invention preferably comprise compounds with similar biological activity to that associated with the B pharmacophore. Non-limiting examples of such pharmacologically active ring systems are shown in FIG. 2.

The libraries of the invention are useful as a source of biologically active compounds. In some preferred embodiments, the library is designed to maximize structural diversity, with there thus being a substantial likelihood that one or more library compounds possesses useful biological activity toward one or more biological targets. In some other preferred embodiments, the library is designed to maximize structural similarity to a compound with known biological activity, with there thus being a substantial likelihood that one or more library compounds possesses similar biological activity toward the target of interest.

The libraries can be used in any application for which it is useful to screen multiple compounds. For example, the libraries have utility in screening assays for compounds of use in the pharmaceutical or agricultural industries. The libraries can be assayed for the discovery of new drugs, herbicides, pesticides, or antimicrobial agents.

The libraries of the invention may be screened to identify which of the component library compounds possess the biological activity of interest. Preferably, the library is designed so as to facilitate the identification of individual library compounds with the desired biological activity. Methods for coding library compounds in a combinatorial library are known to those of skill in the art. Nonlimiting examples for coding methods include radiofrequency tags, nucleic acid tags, colorimetric tags, spectrometric tags (Raman, IR), polyhalogenated benzene tags, and polymorph tags. In certain preferred embodiments, the library compounds are mass-coded, and are distinguished on the basis of their mass spectra.

The libraries may be screened by any method that is appropriate for the biological target and compatible with the method used to deconvolute the mixture and identify individual active library compounds. The screening assay may be a binding assay, wherein compounds that bind to the target are separated from compounds that do not bind to the target, or it may be a functional assay, wherein the effect of the library on a function of target is measured. The concentration of the library will depend on the sensitivity of the instrument, for example, a mass spectrometer, used to detect hits. Typically, the library is used in sufficient concentration that each compound in the library is present at a concentration from about 1.0 nM to about 0.1 mM.

In certain preferred embodiments, a target biomolecule, such as a protein, is contacted with the library of the invention so that biomolecule-ligand complexes form between the biomolecule and any library compounds that are ligands for the biomolecule. Compounds that do not bind the biomolecule are separated from the biomolecule-ligand complexes. The biomolecule-ligand complexes are then dissociated, the ligands are separated, and the identity of the ligands is determined.

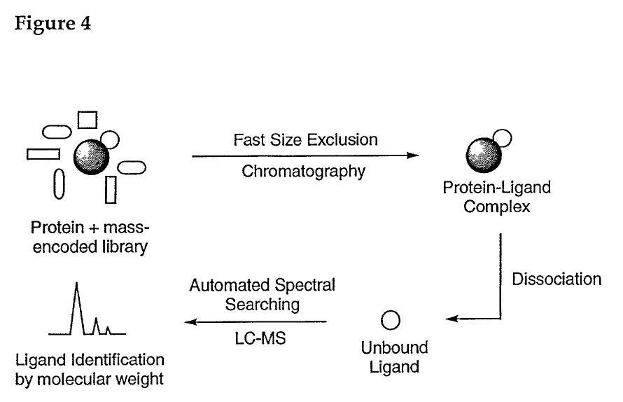

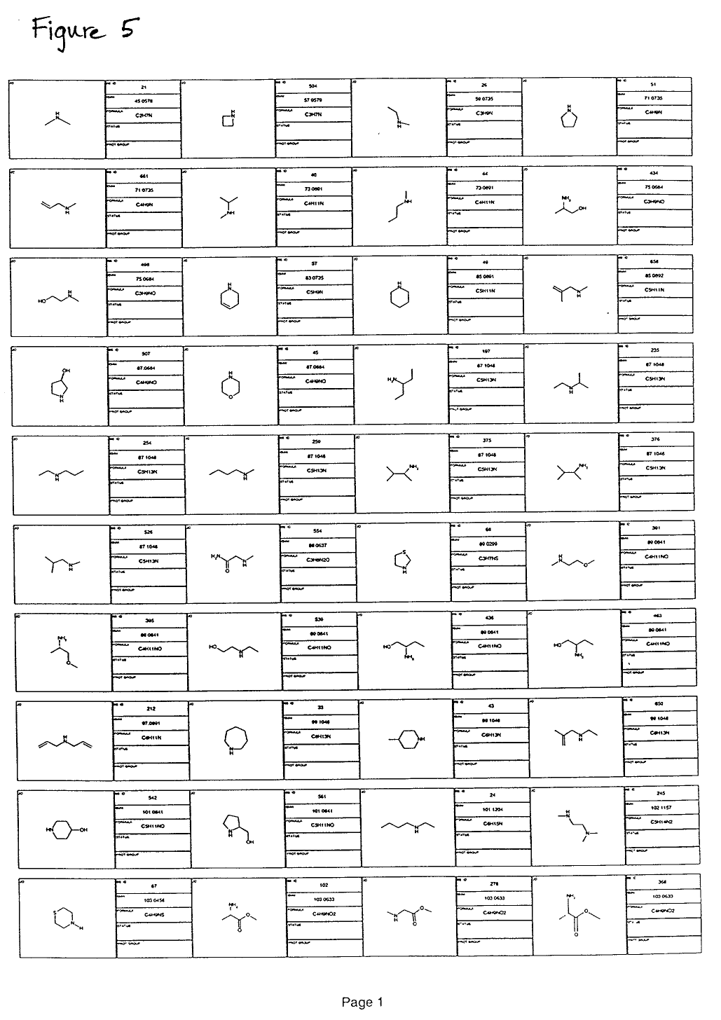

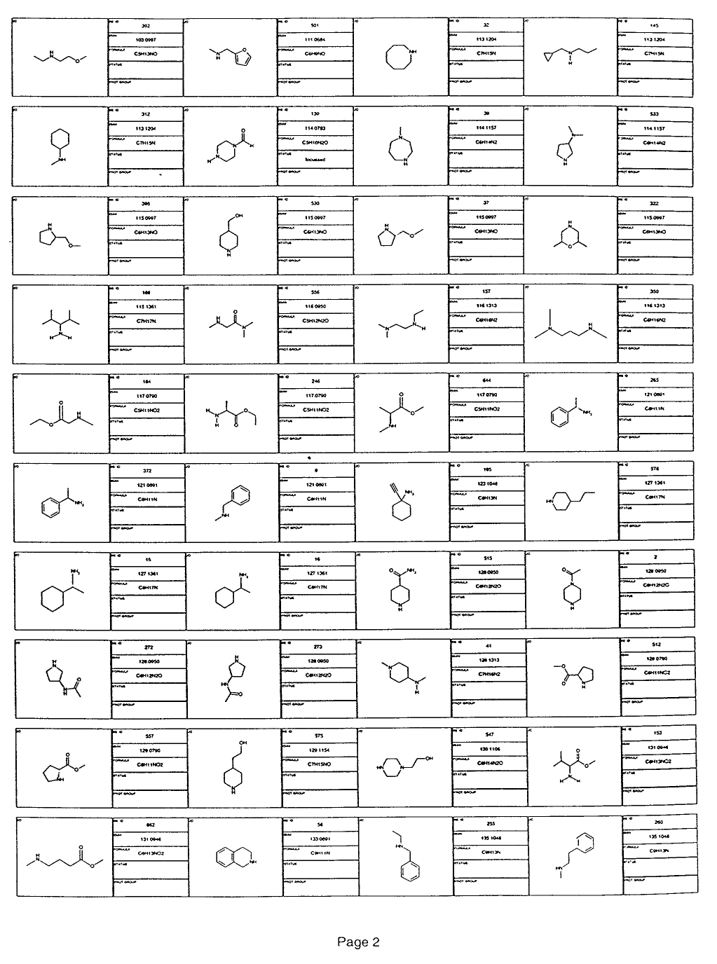

In some preferred embodiments, the library compounds are mass-coded, and are identified by their molecular masses. FIG. 4 is a schematic representation of one such preferred embodiment. Combining a protein and a mass-encoded small molecule library in a physiologically relevant buffer leads to the formation of complexes of the protein with any library compounds that are ligands for the protein. These complexes are preferably separated from non-binding library members by rapid, low temperature (<10 seconds at 0° C.) size exclusion chromatography (SEC). In contrast to other affinity selection methods, such as spin column (Huyer et al., Anal. Biochem. 1998, 258, 19-30) or ultrafiltration (Zhao et al., J. Med. Chem. 1997, 40, 4006-4012), a rapid SEC separation insures that even weakly bound ligands are captured for identification as possible lead structures. The SEC band containing the protein-ligand complexes preferably is immediately analyzed by LC/MS under conditions (e.g., acid, basic, chemical-denaturing) such that ligands dissociate from the protein. In some preferred embodiments, the liquid chromatography step is performed on a reverse-phase chromatography column, which is preferably maintained at 60° C. to promote dissociation of ligands from the complex. The ligand is eluted into a high-resolution mass spectrometer for analysis; automated software algorithms search the mass spectral data to identify ligands by virtue of their molecular weight.

Preferably, a liquid chromatography/mass spectroscopy (LC/MS) analysis of the protein itself (i.e., the protein that has not been exposed to the library) is first performed to provide a background value. The background value is used to eliminate chemical noise, including noise resulting from protein breakdown products, contaminated solvents and buffers, machine contamination, and previous chemicals used in the LC/MS, as well as system electronic noise. The SEC band containing the protein-ligand complexes is then analyzed by LC/MS under conditions (e.g., acid, basic, chemical-denaturing) such that ligands dissociate from the protein. This output is compared to the background at each mass-to-charge (m/z) value corresponding to a library compound. If the expected ligand signal is above the measured background level, a possible hit is recorded. By matching the empirically obtained mass values and calculated mass values, the molecular weights of library molecules that are able to bind to the target protein are deduced. Because this method directly identifies the compound of interest, the incidence of false positives is very low.

Ideally, the structures of the library molecules of interest are deduced directly from their molecular weights. However, if the structure of a library molecule is not uniquely determined by its molecular weight, an iterative process is used to identify the structure of the biologically active library molecule. The iterative process involves generating a second combinatorial library using only those building blocks which the mass spectroscopy data indicate form one of the structures that corresponds to a hit mass value. As a result, the second library contains the possible hit structure in a much smaller combinatorial library, a factor which facilitates mass spectroscopy analysis. The second library is screened and subjected to mass spectroscopy. The empirical mass values are compared to the theoretic mass values (as described above) to deduce the structures of the library molecules of interest. This process is repeated until there is sufficient molecular weight data to determine the structure of the biologically active library molecule.

Certain particularly preferred methods for screening libraries and identifying active library compounds are described in Nash et al., WO 99/35109, and in Birnbaum et al., WO 00/22649, each of which is hereby incorporated by reference in its entirety.

In a third aspect, the invention provides a compound of the formula

A-B-C,

wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an epoxide functional group.

The compounds of the invention are useful as core molecules for the preparation of combinatorial libraries, as described above. The compounds of the invention are also useful as cross-linking reagents. Cross-linking reagents have two or more reactive groups connected by a linker. In the compounds of the invention, A and C comprise reactive groups, and B is a linker connecting the reactive groups. Cross-linking reagents may be used to join two molecules or two remote portions of the same molecule. For example, cross-linking reagents are useful for conjugation or immobilization of proteins and nucleic acids.

Cross-linking reagents are useful in the study of biological macromolecules, by enabling the selective or site-specific chemical cross-linking of one macromolecule to another molecule. The reactive centers of the cross-linking reagent may either be chemically equivalent (homofunctional cross-linking reagents) or chemically different (heterofunctional cross-linking reagents). Homofunctional agents enable the cross-linking of molecules that possess the same nucleophilic moieties, while heterofunctional agents enable the selective cross-linking of molecules that contain different nucleophilic moieties.

To identify biological macromolecules that act by binding non-covalently to each other, researchers have used chemical cross-linking agents to trap the non-covalent interaction as a stable, covalently bound species that is amenable to further study. Researchers have also used cross-linking agents to covalently immobilize biological macromolecules on solid surfaces for applications such as affinity chromatography and immunological assays, including ELISA assays. Researchers have also used cross-linking reagents to covalently attach fluorescent or radiolabeled small molecules to the biological macromolecule for use in diagnostic imaging applications. The use of cross-linking reagents is further described, e.g., by Mattson et al., Mol. Biol. Repts., 17: 167-183 (1993), and by Hermanson, Bioconjugate Techniques, Academic Press: New York, 1996, p. 728.

The term “combinatorial library”, as used herein, refers to a collection of compounds that is synthesized from combinations of two or more starting components. At least some of the compounds must differ from at least some of the other compounds in the library. A library preferably includes at least 100 compounds, and more preferably includes at least 500, 1000, 5000, or more compounds.

The term “library compounds”, as used herein, refer to the molecules that are in a combinatorial library. Library compounds are the products of reactions between cores and building blocks.

The term “deconvolute”, as used herein, refers to a process of determining which compound in a mixture is responsible for an observed activity.

The term “cores”, as used herein, refer to molecules having at least two reactive centers that react with functional groups of building blocks. The terms “core”, “core molecule”, and “core compound” all refer to these molecules, and are used interchangeably. The term “dicore” refers to a core molecule having two reactive centers. The term “tricore” refers to a core molecule having three reactive centers. The term “tetracore” refers to a core molecule having four reactive centers.

A “reactive center”, as used herein, refers to a functional group of a core that is capable of forming a linkage (e.g., a covalent bond) with a complementary functional group of a building block. For example, an electrophilic reactive center, including without limitation an acid halide or activated ester, is capable of forming a linkage with a complementary nucleophilic building block functional group, including without limitation an amine, hydroxy, or thiol functional group.

The term “building blocks”, as used herein, refer to molecules having at least one functional group that can react with the reactive centers of core molecules to form combinatorial library molecules.

A “rotatable bond”, as used herein, refers to a bond about which rotation can occur such that the relative spatial orientation of reactive centers on a core changes or the relative spatial orientation of building block moieties bonded to a core moiety changes.

“Spatial orientation”, as used herein, refers to the placement in space of at least two moieties (attached to the same molecule, such as a core) relative to one another.

The term “organic moiety”, as used herein, refers to a group having from 1 to about 25 carbon atoms; from 0 to about 10 heteroatoms selected from the group consisting of oxygen, nitrogen, and sulfur; and from 0 to about 6 halogen atoms.

A “carbocyclic ring”, “carbocyclic group” or “carbocycle”, as used herein, is a ring structure having from 3 to about 8, preferably 3, 5, or 6 carbon atoms, any of which atoms may be optionally substituted. Some carbocyclic rings are saturated or partially unsaturated. Such saturated or partially unsaturated carbocyclic rings include, without limitation, cyclopropyl, cyclopentyl, cylohexyl, cylopentenyl, cyclohexenyl, and cyclohexadienyl. Some other carbocyclic rings are aromatic rings.

A “heterocyclic ring”, “heterocylic group”, or “heterocycle” is a ring structure having from 3 to about 8 atoms, wherein one or more atoms are selected from the group consisting of N, O, and S. The heterocyclic ring may be optionally substituted on carbon at one or more positions. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Some heterocylic rings are saturated or partially unsaturated. Examples of saturated heterocyclic rings include, without limitation, epoxide, aziridine, tetrahydrofuran, pyrrolidine, piperidine, piperazine, thiazolidine, oxazolidine, oxazolidinone, and morpholine. Some other heterocyclic rings are aromatic rings.

An “aromatic ring” or “aryl group”, as used herein, comprises 5 or 6 ring atoms, and has 6π electrons shared in a cyclic array. The terms “aromatic ring” and “aryl group” are intended to include heteroaryl groups, which have, in addition to carbon atoms, from one to about four, preferably one or two, heteroatoms selected from the group consisting of N, O, and S. Examples of aromatic rings include, without limitation, benzene, pyridine, pyrimidine, pyrazine, thiophene, furan, pyrrole, imidazole, pyrazole, oxazole, isoxazole, thiazole, triazole, and tetrazole.

The aromatic ring may be fused to one or more other aromatic rings to form a fused aromatic ring system, comprising 5 to 14 ring atoms, preferably 5, 6, 9, or 10 ring atoms, and having 6, 10, or 14π electrons shared in a cyclic array. The aromatic ring may also be fused to one or more non-aromatic rings. Examples of fused ring systems include, without limitation, benzofuran, benzothiophene, quinoline, isoquinoline, quinoxaline, tetrahydroquinoline, dibenzofuran.

The term “activated ester group”, as used herein, refers to a —C(O)OR group, wherein OR is a good leaving group. A good leaving group is an organic moiety that can readily stabilize a negative charge. Preferably, R contains at least one electron withdrawing group. Electron withdrawing groups are well-known to those of skill in the art and include, without limitation carbonyl, chloro, fluoro, and nitro. More preferably, R is imido, haloalkyl, or aryl substituted with at least one electron withdrawing substituent.

An “isocyanate equivalent”, as used herein is a —NH—C(O)OR moiety, wherein OR is a good leaving group, as described above.

As employed herein, a “substituted” alkyl, aryl, heteroaryl, carbocyclic or heterocyclic group is one having from one to about four, preferably from one to about three, more preferably one or two, non-hydrogen substituents. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, thiol, alkanesulfonyl, arenesulfonyl, alkanesulfonamido, arenesulfonamido, aralkylsulfonamido, acyl, acyloxy, cyano, oximino, and ureido groups.

The term “halogen” or “halo” as employed herein refers to chlorine, bromine, fluorine, or iodine.

As herein employed, the term “acyl” refers to an alkylcarbonyl or arylcarbonyl substituent.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. Unless otherwise explicitly limited, the term “amino” is meant to include NH₂, alkylamino, dialkylamino, arylamino, aralkylamino, and cyclic amino groups.

As used herein, the term “amine” refers to a compound having at least one amino group. Amine building blocks preferably have primary or secondary amino groups, i.e., the amino group is preferably NH₂ or alkylamino.

The term “oximino” refers to a ═N(OH) or ═N(OR) group, wherein R is alkyl, aryl, aralkyl, sulfonyl, or acyl. Unless otherwise explicitly limited, the term “oximino” is meant to include oximes of either E- or Z-configuration, or mixtures thereof.

The term “ureido” as employed herein refers to a substituted or unsubstituted urea moiety.

The term “pharmacophore”, as used herein, refers to a chemical moiety that is associated with a particular pharmacological activity.

The present invention will now be illustrated by the following examples, which are not intended to be limiting in any way.

### EXAMPLES

### Example 1

### Test Protocol for Determining Building Block Suitability

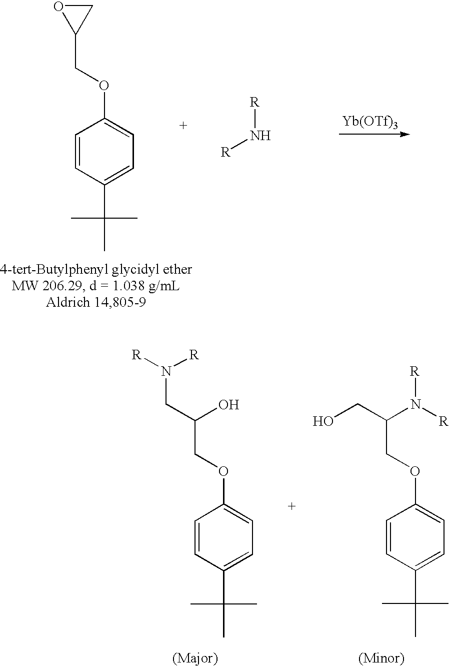

Green-ribbed vials were heated in an oven, cooled in a dessicator, and flushed with argon immediately prior to use. These vials were used for preparation of stock solutions, as well as for the reactions.

An Yb(OTf)₃ stock solution was prepared by dissolving 120 mg in distilled THF (1.5 mL). Sonication and gentle vortexing was required to maximize solubility.

Dichloromethane (DCM) (1.2 mL, from a newly opened bottle) was added to the reaction vial, followed by the amine (0.18 mmol). The Yb(OTf)₃ stock solution (150 μL) was added, followed by the epoxide (0.15 mmol, 31 mg, 30 μl). The vial was flushed with inert gas and the vial cap was tightened. Each sample was then sonicated (20-30s) and checked by thin layer chromatography (TLC) (elution with 97:2:1 DCM:MeOH:TEA) periodically, starting at 2 hours, and then every 6-8 hours). The product(s) had Rf values of 0.1-0.2, the starting material had an R_{f} value of >0.9, and a trace core-based impurity at an R_{f} value of 0.6 was also be present.

Upon completion of the reaction, each sample was scavenged with isocyanate resin (100 mg), stirred for 30 min, and then filtered through glass wool-packed pipettes into pre-weighed green-ribbed vials. The product solutions were analyzed by HPLC using an ELSD detector. Samples for mass spectral analysis were prepared at a concentration of 5-10 ng/mL. All reactions in which a single peak (not matching the retention time of the starting epoxide or amine) accounted for >80% of the HPLC area above noise level, and whose mass spectrum matched that predicted for the desired amino alcohol were deemed to have “passed” this quality control (QC) protocol.

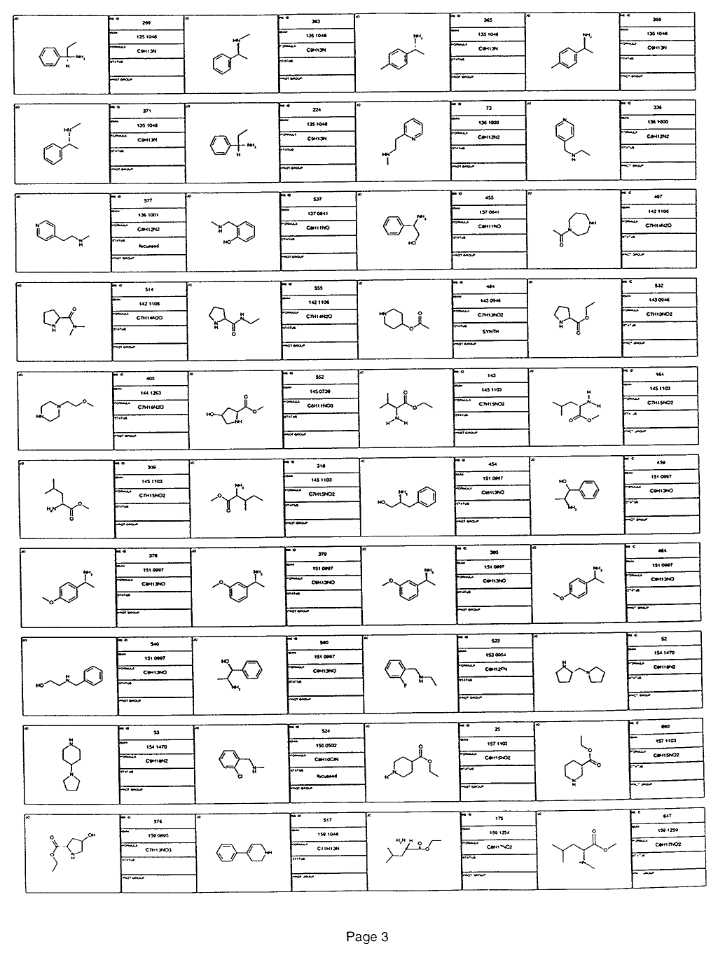

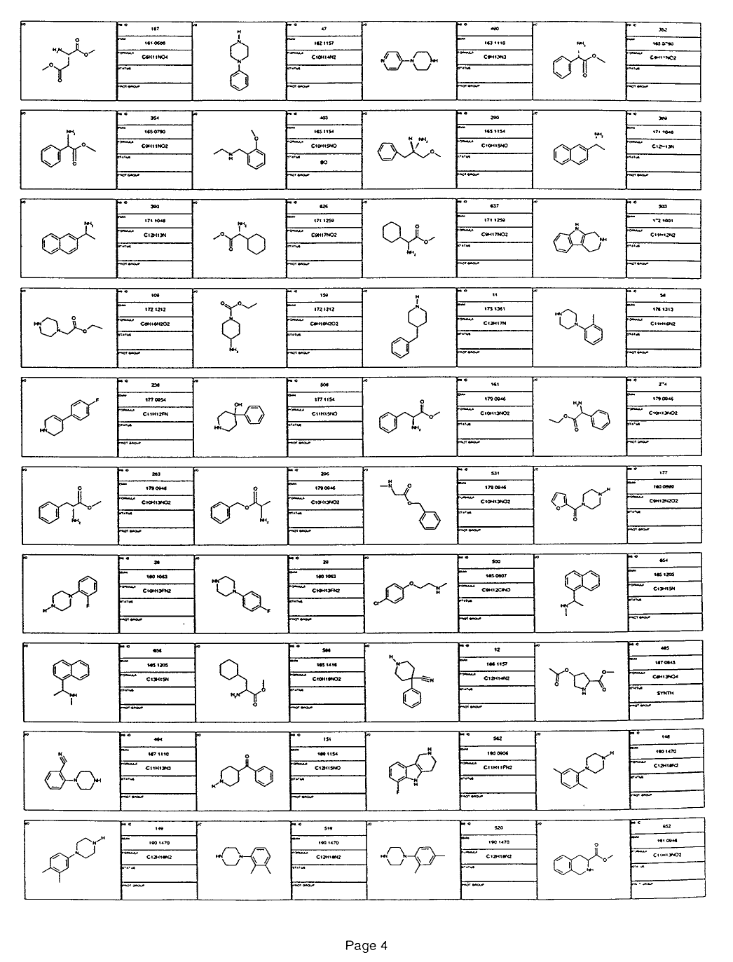

After confirming that individual amines would each react to form a pure compound, other experiments were conducted to show that the amines were competitive in reactivity. The experiments were run with representative sets of two to five amines. The mass spectra of the product mixtures showed distinct masses corresponding to the desired compounds. Analysis of the QC results resulted in a list of compatible amine building blocks, as shown in FIG. 5.

### Example 2

### Synthesis of Core 2

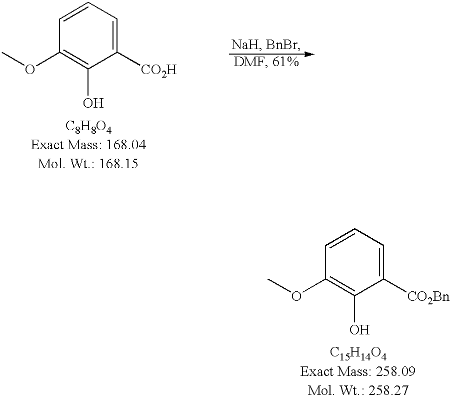

### Benzyl 2-hydroxy-3-methoxybenzoate

To 2-hydroxy-3-methoxybenzoic acid (10.0 g, 60 mmol) in DMF (50 mL) was added 60% NaH (2.64 g, 66 mmol) at 0-5° C. The ice bath was removed after 15 minutes and the reaction mixture was stirred for 1 hour. Benzyl bromide (7.2 mL, 60 mmol) was added to the mixture and it was stirred overnight at room temperature. The crude mixture was poured into water (200 mL) and extracted with 25% ethyl acetate/hexanes. The combined organics were washed with saturated sodium bicarbonate and brine, dried over sodium sulfate and concentrated. Purification by column chromatography (SiO₂, 5% ethyl acetate /hexanes) afforded an off-white solid (9.46 g, 61%).

¹H NMR (300 MHz, CDCl₃) δ10.97 (s, 1H), 7.40 (m, 6H), 7.05 (d, 1H, J=8.0 Hz 6.81 (apparent t, 1H, J=8.3 Hz), 5.39 (s, 2H), 3.90 (s, 3H). MS calcd for C₁₅H₁₅O₄ [M+H]⁺259.097, found 259.109.

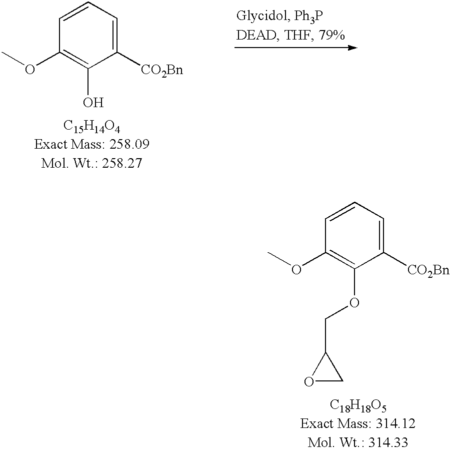

### Glycidol Ether Benzyl Ester

To a mixture of benzyl 2-hydroxy-3-methoxy-benzoate (9.46 g, 36.6 mmol), triphenylphosphine (13.4 g, 51.4 mmol) and glycidol (3.4 mL, 51.3 mmol) in THF (150 mL) was added DEAD (8.1 mL, 51.3 mmol). The reaction mixture was stirred for 4 hours before being concentrated to a pale yellow solid. Purification by column chromatography (SiO₂, 1% MeOH/DCM) afforded a clear oil (9.08 g, 79%).

¹H NMR (300 MHz, CDCl₃) δ7.45 (m, 2H), 7.38 (m, 4H), 7.05 (m, 2H), 5.35 (s, 2H), 4.13 (dd, 1H, J=4.1, 10.8 Hz), 4.04 (dd, 1H, J=5.7, 10.7 Hz), 3.85 (s, 3H), 3.28 (m, 1H), 2.74 (apparent t, 1H, J=4.6 Hz), 2.58 (dd, 1H, J=2.7, 5.0 Hz).

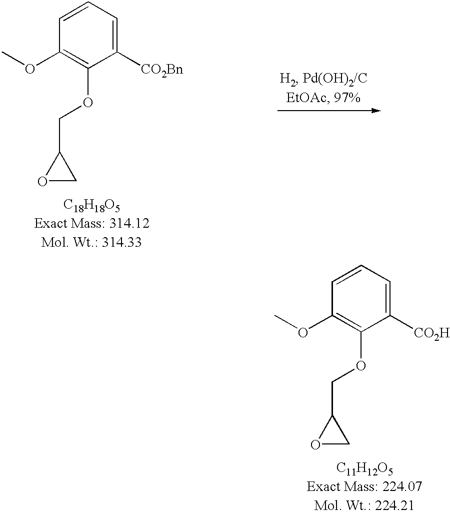

### Glycidol Ether Carboxylic Acid

A mixture of benzyl ester (7.84 g, 24.7 mmol) and 20% palladium hydroxide on carbon (800 mg) in ethyl acetate (120 mL) was evacuated and flushed with hydrogen (balloon). After 1 hour the mixture was filtered through a pad of Celite and concentrated to afford an off-white solid (5.38 g, 97%). The material contained a small amount of reduced epoxide with a characteristic doublet at 1.26 ppm (3H).

¹H NMR (300 MHz, CDCl₃) δ7.73 (dd, 1H, J=0.7, 6.5 Hz), 7.14 (m, 2H), 4.46 (dd, 1H, J=3.2, 11.2 Hz), 4.28 (dd, 1H, J=5.8, 11.1 Hz), 3.91 (s, 3H), 3.40 (m, 1H), 2.92 (apparent t, 1H, J=4.6 Hz), 2.75 (dd, 1H, J=2.5, 4.7 Hz).

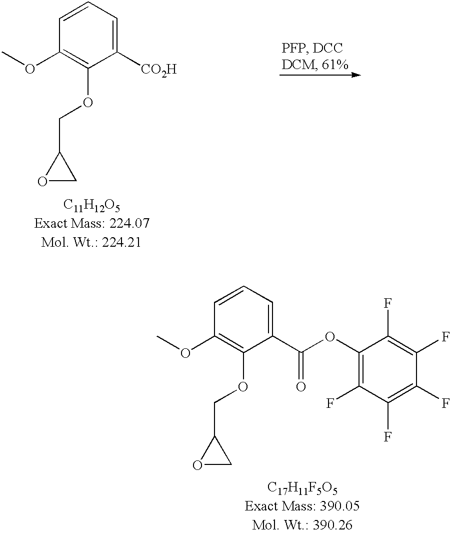

### Core 2

To the epoxy acid (1.00 g, 4.5 mmol) in DCM (30 mL) was added a solution of pentafluorophenol (PFP) (0.9 g, 4.9 mmol) and 1M dicyclohexylcarbodiimide (DCC) (4.9 mL, 4.9 mmol) in DCM (5 mL). The reaction mixture was stirred for 3 hours before being cooled in the freezer for 1 hour. The white precipitate was removed by filtration and the filtrate was concentrated to a viscous orange oil. Purification by column chromatography (SiO₂, 30% ethyl acetate/hexanes) afforded the product as a white solid (1.07 g, 61%) and the reduced epoxide product as a clear oil (132 mg).

¹H NMR (300 MHz, CDCl₃) δ7.59 (apparent t, 1H, J=4.7 Hz), 7.20 (dd, 2H, J=4.7 Hz), 4.26 (dd, 1H, J=3.8, 10.7 Hz), 4.14 (dd, 1H, J=5.8, 10.8 Hz), 3.91 (s, 3H), 3.40 (m, 1H), 2.82 (apparent t, 1H, J=4.6 Hz), 2.66 (dd, 1H, J=2.5, 4.8 Hz). MS (+, 30 V) calcd for C₁₇H₁₁F₅O₅ [M+H]⁺391.053, found 391.358.

### Example 3

### Synthesis of Core 3

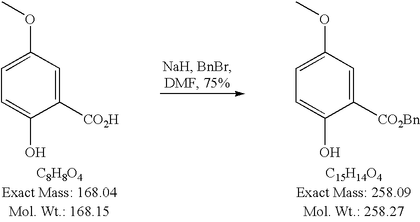

### Benzyl 2-hydroxy-5-methoxybenzoate

To 2-hydroxy 5-methoxybenzoic acid (5.0 g, 30 mmol) in DMF (30 mL) in an ice bath was added 60% NaH (1.32 g, 33 mmol). The reaction mixture was stirred for 15 minutes before warming to room temperature and stirring for 1 hour. Benzyl bromide (3.6 mL, 30 mmol) was added, and the reaction mixture was stirred overnight. The reaction mixture was poured into water (100 mL) and extracted with 25% ethyl acetate/hexanes. The combined organics were washed with water and brine, dried over sodium sulfate and concentrated to a yellow oil. Purification by column chromatography (SiO₂, 5% ethyl acetate/hexanes) afforded an oil (5.8 g, 75%).

¹H NMR (300 MHz, CDCl₃) δ10.35 (s, 1H), 7.38 (m, 6H), 7.09 (dd, 1H, J=3.1, 9.0 Hz), 6.93 (d, 1H, J=9.0 Hz), 5.40 (s, 2H), 3.77 (s, 3H). MS calcd for C₁₅ H₁₅O₄ [M+H]⁺259.097, found 259.090.

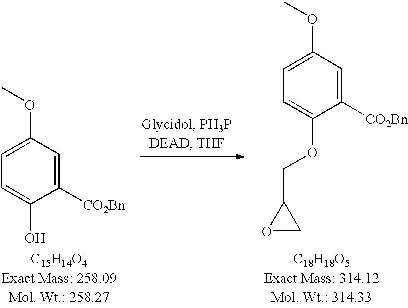

### Glycidol Ether Benzyl Ester

To a mixture of benzyl 2-hydroxy-3-methoxy-benzoate (5.8 g, 22.5 mmol), triphenylphosphine (8.23 g, 31.4 mmol) and glycidol (2.1 mL, 31.4 mmol) in THF (100 mL) was added diethyl azodicarboxylate (DEAD) (6.2 mL, 31.4 mmol). The reaction mixture was stirred for 21 hours before being concentrated. Purification by column chromatography (SiO₂, 1% MeOH/DCM) afforded starting material (2.26 g, 39%) a mix of starting material and product (1.6 g) and product (2.41 g, 33%).

¹H NMR (300 MHz, CDCl₃) δ7.46 (m, 2H), 7.36 (m, 3H), 6.99 (m, 2H), 5.36 (s, 2H), 4.19 (dd, 1H, J=3.2, 11.0 Hz), 4.00 (dd, 1H, J=5.7, 11.0 Hz), 3.78 (s, 3H), 3.26 (m, 1H), 2.78 (apparent t, 1H, J=4.9 Hz), 2.73 (dd, 1H, J=2.1, 4.9 Hz).

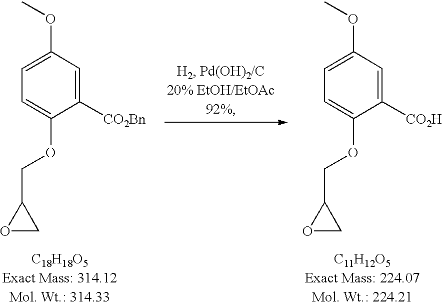

### Glycidol Ether Carboxylic Acid

A mixture of the benzyl ester (3.94 g, 12.4 mmol) and 20% palladium hydroxide on carbon (400 mg) in 20% ethanol/ethyl acetate (100 mL) was evacuated and flushed with hydrogen (balloon). After 1 hour the mixture was filtered through a pad of Celite and concentrated to afford a green solid (2.58 g, 92%).

¹H NMR (300 MHz, CDCl₃) δ7.67 (d, 1H, J=3.1), 7.10 (dd, 1H, J=3.1, 9.0 Hz), 7.02 (d, 1H, J=9.0 Hz) 4.50 (dd, 1H, J=2.7, 11.2 Hz), 4.16 (dd, 1H, J=5.6, 11.2 Hz), 3.82 (s, 3H), 3.40 (m, 1H), 2.97 (apparent t, 1H, J=4.4 Hz), 2.80 (dd, 1H, J=2.6, 4.6 Hz).

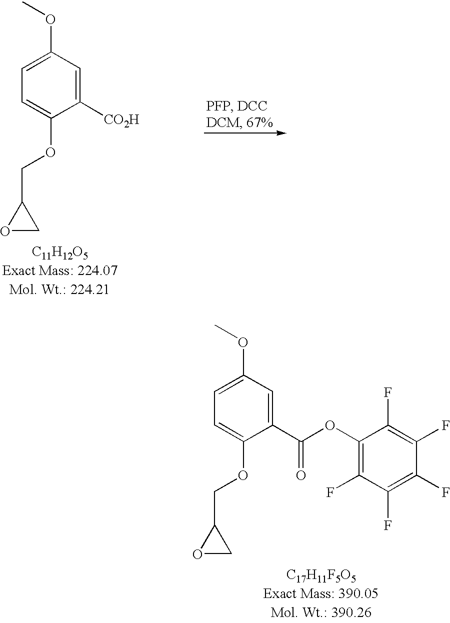

### Core 3

To the epoxy acid (3.14 g, 14 mmol) in DCM (100 mL) was added a solution of PFP (3.1 g, 16.8 mmol) and 1M DCC (17 mL, 17 mmol) in DCM (10 mL). The reaction mixture was stirred for 3 hours before being cooled in the freezer for the weekend. The white precipitate was removed by filtration and the filtrate was concentrated. Purification by column chromatography (SiO₂, 30% ethyl acetate /hexanes) afforded the product as a white solid (3.69 g, 67%).

¹H NMR (300 MHz, CDCl₃) δ7.53 (d, 1H, J=1.6 Hz), 7.16 (dd, 2H, J=1.6, 9.1 Hz), 7.03 (d, 1H, J=9.1 Hz), 4.31 (d, 1H, J=11.0 Hz), 4.07 (dd, 1H, J=1.2, 11.1 Hz), 3.83 (s, 3H), 3.36 (m, 1H), 2.86 (m, 2H). MS (+, 30 V) calcd for C₁₇H₁₁F₅O₅ [M+H]⁺ 391.053, found 391.358.

### Example 4

### Synthesis of Core 4

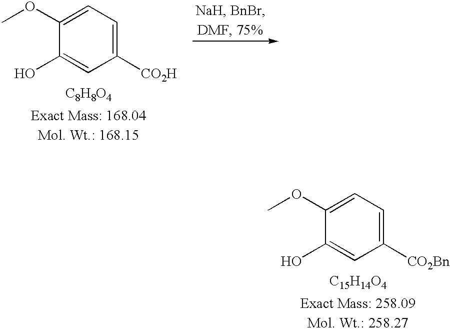

### Benzyl 3-hydroxy-4-methoxybenzoate

To 3-hydroxy-3-methoxybenzoic acid (10.0 g, 59 mmol) in NMP (40 mL) was added 60% NaH (2.6 g, 65 mmol). The reaction mixture was stirred at 40° C. for 1 hour.

Benzyl bromide (8 mL, 65 mmol) was added, and the reaction mixture was stirred overnight at 40° C. The reaction mixture was poured into water (500 mL) and extracted with 25% ethyl acetate/hexanes. The combined organics were washed with water, saturated sodium bicarbonate solution and brine, dried over sodium sulfate and concentrated to an oil. Purification by column chromatography (SiO₂, 20% ethyl acetate/hexanes) afforded a white solid (9.87 g, 64%).

¹H NMR (300 MHz, CDCl₃) δ7.63 (m, 2H), 7.38 (m, 5H), 6.87 (d, 1H, J=8.3 Hz), 5.62(s, 1H), 5.33 (s, 2H), 3.95 (s, 3H). MS calcd for C₁₅H₁₅O₄ [M+H]⁺259.097, found 259.071.

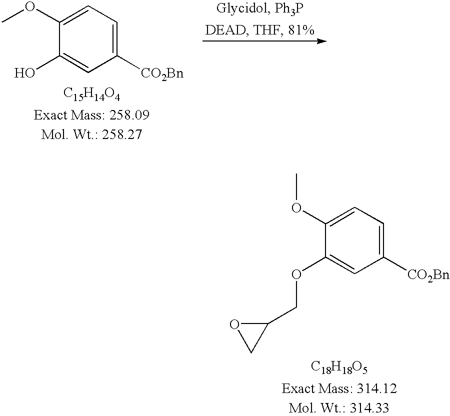

### Glycidol Ether Benzyl Ester

To a mixture of benzyl 3-hydroxy-4-methoxy-benzoate (9.87 g, 38.2 mmol), triphenylphosphine (14 g, 53.5 mmol) and glycidol (3.6 mL, 53.5 mmol) in THF (150 mL) was added DEAD (8.4 mL, 53.5 mmol). The reaction mixture was stirred for 7 hours. Thin layer chromatography (TLC) showed the reaction to be incomplete so an additional 0.2 equivalents of triphenylphosphine (2 g, 7.64 mmol), glycidol (0.5 mL, 7.64 mmol) and DEAD (1.2 mL, 7.64 mmol) were added to the mixture. After 1.5 hours the mixture was concentrated to an off-white solid. Purification by column chromatography (SiO₂, 1% MeOH/DCM) afforded a solid (9.87 g, 81%).

¹H NMR (300 MHz, CDCl₃) δ7.75 (dd, 1H, J=2.1, 8.7 Hz), 7.62 (d, 1H, J=2.0 Hz), 7.40 (m, 5H), 6.89 (d, 1H, J=8.5 Hz), 5.34 (s, 2H), 4.29 (dd, 1H, J=3.5, 11.3 Hz), 4.06 (dd, 1H, J=5.6, 11.3 Hz), 3.40 (m,1H), 2.90 (app, t, 1H J=4.5 Hz), 2.76 (dd, 1H, J=2.6, 4.9 Hz).

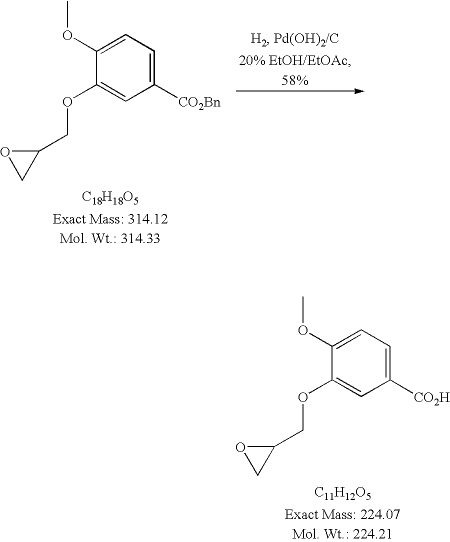

### Glycidol Ether Carboxylic Acid

A mixture of benzyl ester (9.1 g, 28.7 mmol) and 20% palladium hydroxide on carbon (910 mg) in 20% ethanol/ethyl acetate (150 mL) was evacuated and flushed with hydrogen (balloon). After 3 hours the mixture was filtered through a pad of Celite and concentrated to afford a green solid (3.71 g, 58%).

¹H NMR (300 MHz, CDCl₃) δ7.81 (d, 1H, J=2.0, 8.6 Hz) 7.64 (d, 1H, J=1.7 Hz), 6.93 (d, 1H, J=8.6 Hz) .4.32 (dd, 1H, J=3.3, 11.3 Hz), 4.16 (dd, 1H, J=5.6, 11.3 Hz), 3.95 (s, 3H), 3.40 (m, 1H), 2.92 (apparent t, 1H, J=4.6 Hz), 2.78 (dd, 1H, J=2.6, 4.9 Hz). MS calcd for C₁₁H₁₁O₅ [M−H]⁻223.062, found 223.006.

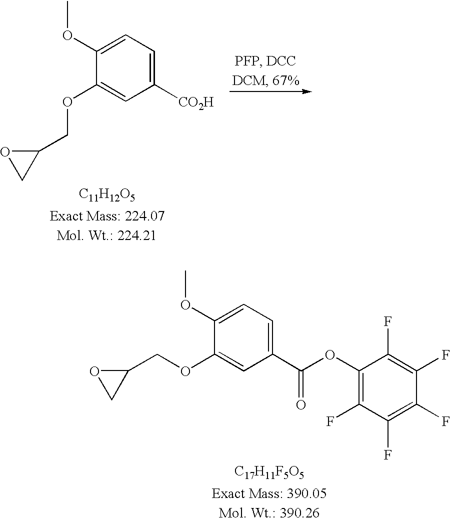

### Core 4

To the epoxy acid (3.71 g, 16.5 mmol) in dioxane/DCM (200 mL) was added a solution of PFP (3.35 g, 18.15 mmol) and IM DCC (18.2 mL, 18.2 mmol) in DCM (10 mL). The reaction mixture was stirred for 3 hours before being cooled in the freezer overnight. The white precipitate was removed by filtration and the filtrate was concentrated. Purification by column chromatography (SiO₂, 20% ethyl acetate /hexanes) afforded the product as a white solid (4.72 g, 73%).

¹H NMR (300 MHz, CDCl₃) δ7.89 (dd, 1H, J=2.0, 8.6 Hz), 7.69 (d, 1H, J=2.0 Hz), 6.98 (d, 1H, J=8.6 Hz), 4.36 (dd, 1H, J=3.2, 11.3 Hz), 4.07 (dd, 1H, J=5.8, 11.3 Hz), 3.97 (s, 3H), 3.42 (m, 1H), 2.93 (apparent t, 1H, J=4.5 Hz), 2.79 (dd, 1H, J=2.6, 4.9 Hz). MS (+, 30 V) calcd for C₁₇H₁₁F₅O₅ [M+H]⁺391.053, found 391.358

### Example 5

### Synthesis of Core 5

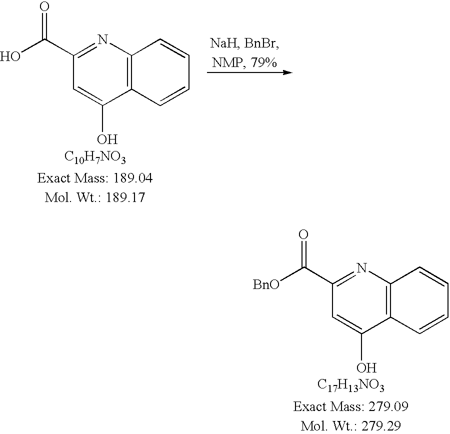

### Benzyl 4-hydroxyquinoline-2-carboxylate

To 4-hydroxyquinoline-2-carboxylic acid (2.08 g. 11 mmol) in N-methyl-pyrrolidinone (NMP) (20 mL) was added 60% NaH (290 mg, 12.1 mmol). The cloudy reaction mixture was heated to 40° C. for 1 hour to afford a clear yellow solution. To the mixture was added benzyl bromide (1.31 mL, 11 mmol) and the reaction mixture was stirred overnight at 40° C. After cooling the reaction mixture was poured into water (100 mL). The resulting precipitate was collected by filtration, washed with 20% ethyl acetate/hexanes (100 mL) and dried to afford the product (2.44 g, 79%).

¹H NMR (300 MHz, DMSO) δ8.08 (d, 1H, J=8.0 Hz), 7.95 (d, 1H, J=8.4 Hz), 7.72 (apparent t, 1H, J=7.0 Hz), 7.52 (d, 1H, J=7.3 Hz), 7.41 (m, 5H), 6.64 (d, 1H, J=9.0 Hz), 5.46 (s, 2H).

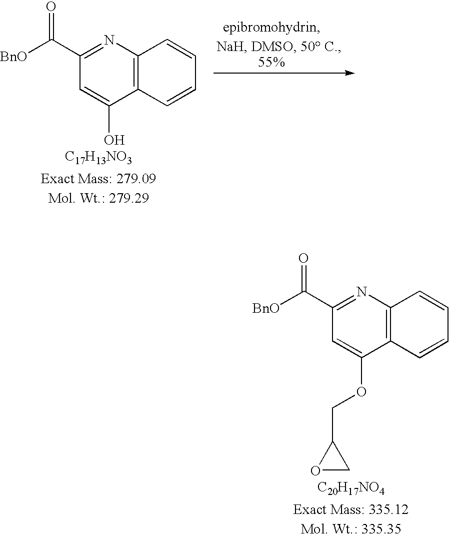

### Core 5 Benzyl Ester

To benzyl 4-hydroxyquinoline-2-carboxylate (1.89 g, 6.76 mmol) in DMSO (20 mL) was added 60% NaH (325 mg, 8.1 mmol). The reaction mixture was heated to 50° C. for 45 minutes before epibromohydrin (1.16 mL, 13.5 mmol) was added. The reaction mixture was stirred 22 hours at 50° C. The mixture was poured into water (75 mL) and extracted with 50% ether/ethyl acetate. The combined organics were washed with water (50 mL), saturated sodium bicarbonate solution and brine, dried over sodium sulfate and concentrated. Purification by column chromatography (SiO₂, 2% MeOH/DCM) afforded the product (1.25 g, 55%).

¹H NMR (300 MHz, CDCl₃) δ8.26 (apparent t, 2H, J=9.5 Hz), 7.77 (apparent t, 1H, J=4.3 Hz), 7.64 (apparent t, 1H, J=4.1 Hz), 7.61 (s, 1H), 7.55 (m, 2H), 7.35 (m, 3H), 5.54 (s, 2H), 4.57 (dd, 1H, J=2.8, 11.1 Hz), 4.21 (dd, 1H, J=6.0, 11.1 Hz), 3.51 (m, 1H), 3.00 (apparent t, 1H, J=4.5 Hz), 2.86 (dd, 1H, J=2.6,4.8 Hz).

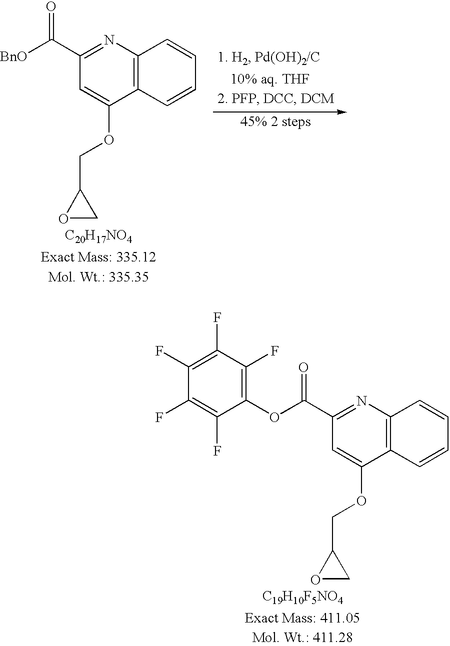

### Core 5

To Core 5 benzyl ester (1.52 g, 4.5 mmol) in 10% aqueous THF (60 mL) under argon was added 20% palladium hydroxide on carbon (150 mg). The mixture was evacuated and flushed with hydrogen (balloon), After 40 minutes the reaction mixture became noticeably cloudy and gray in color. After 3 hours, methanol (60 mL) was added to the mixture in an attempt to dissolve the product. The THF was removed and more methanol (100 mL) was added, but the reaction mixture was still cloudy. The reaction mixture was concentrated and used directly in the next reaction.

To the crude Core 6 acid in NMP (10 mL) was added a mixture of PFP (920 mg, 5 mmol) and 1M DCC (5 mL, 5 mmol) in DCM (5 mL). The mixture was stirred for 30 minutes and filtered through Celite. The Celite was washed with ethyl acetate. The filtrate was poured into water (100 mL) and extracted with 50% ether/ethyl acetate. The combined organics were washed with brine solution, dried and concentrated. An attempt to purify the mixture by column chromatography (SiO₂, 10% hexanes/DCM) did not afford clean fractions (1.38 g). The crude fractions were recrystallized from 30% ethyl acetate/hexanes (40 mL) to afford Core 5 as a white solid (827 mg, 45%).

¹H NMR (300 MHz, CDCl₃) δ8.34 (d, 1H, J=8.0 Hz), 8.29 (d, 1H, J=8.3 Hz), 7.83 (apparent t, 1H, J=7.1 Hz), 7.71 (d, 1H, J=7.1 Hz), 7.66 (s, 1H), 4.66 (dd, 1H, J=2.6 11.1 Hz), 4.26 (dd, 1H, J=6.1, 11.1 Hz), 3.54 (m, 1H), 3.02 (apparent t, 1H, J=4.5 Hz), 2.88 (dd, 1H, J=2.5, 4.7 Hz). MS calcd C₁₉H₁₁F₅NO₄ [M+H]⁺412.058 found 412.163

### Example 6

### Synthesis of Core 6

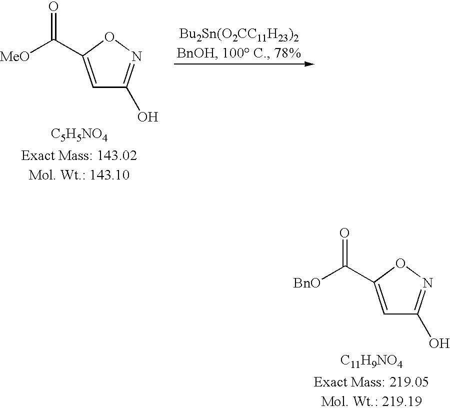

### Benzyl 3-hydroxy-5-isoxazole Carboxylate

Methyl 3-hydroxy-5-isoxazole carboxylate (8.28 g, 57.8 mmol) was heated with benzyl alcohol (30 mL) and dibutyltin dilaurate (1.94 mL, 3.3 mmol, 25 wt %) at 100° C. for 18 hours, according to the procedure described by Leon et al. (J. Am. Chem. Soc. 1996, 118, 8847-8859). The benzyl alcohol was removed in vacuo to afford a rusty brown solid (12.68 g). The solid was dissolved in DCM (100 mL) and washed with water (2×100 mL) and brine (2×100 mL). The organics were dried over sodium sulfate and concentrated. Recrystallization from 30% ethyl acetate/hexanes afforded a pale tan solid (9.92 g, 78%).

¹H NMR (300 MHz, CDCl₃) δ7.40 (m, 5H), 6.60 (s, 1H) 5.38 (s, 2H). MS calcd for C₁₁H₁₀NO₄ [M+H]⁺220.057, found 220.058.

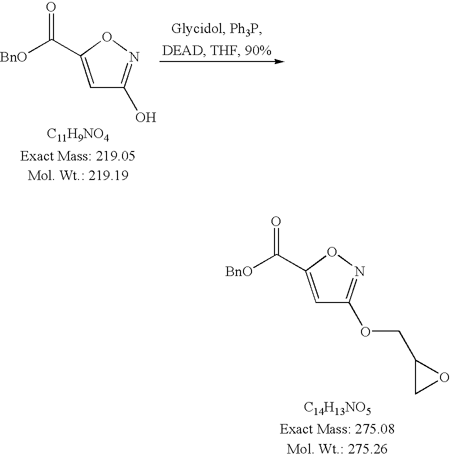

### Core 6 Benzyl Ester

To benzyl 3-hydroxy-5-isoxazole carboxylate (5.06 g, 23.1 mmol) in THF (100 mL) was added triphenylphosphine (7.26 g, 27.7 mmol), glycidol (1.84 mL, 27.7 mmol) and DEAD (4.36 mL, 27.7 mmol). The reaction mixture was stirred for 1 hour and then concentrated. Purification by column chromatography (SiO₂, 1% MeOH/DCM) afforded a yellow oil (5.72 g, 90%).

¹H NMR (300 MHz, CDCl₃) δ7.40 (m, 5H), 6.58 (s, 1H) 5.37 (s, 2H), 4.60 (dd, 1H, J=2.8, 11.8 Hz), 4.16 (dd, 1H, J=6.3, 11.8 Hz), 3.37 (m, 1H), 2.90 (apparent t, 1H, J=4.6 Hz), 2.73 (dd, 1H, J=2.5, 4.7 Hz).

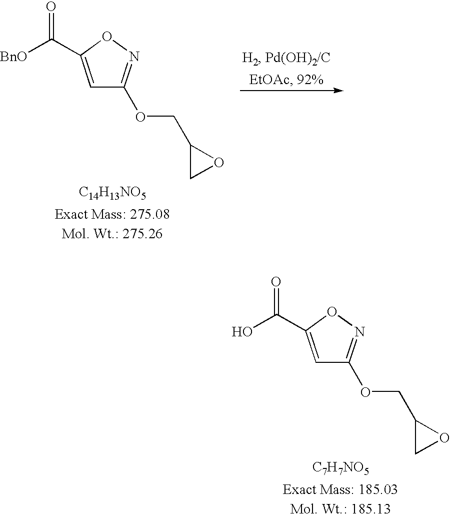

### Core 6 Carboxylic Acid

To Core 6 benzyl ester (1.00 g, 3.63 mmol) in ethyl acetate (100 mL) under argon was added 20% palladium hydroxide on carbon (100 mg). The reaction mixture was evacuated and flushed with hydrogen (balloon). The reaction was stirred for 1 hour, filtered through Celite and concentrated to afford an off-white solid (617 mg, 92%).

¹H NMR (300 MHz, DMSO) δ6.97 (s, 1H), 4.59 (dd, 1H, J=2.4, 11.7 Hz), 4.04 (dd, 1H, J=6.9, 11.7 Hz), 3.35 (m, 1H), 2.85 (apparent t, 1H, J=4.5 Hz), 2.72 (dd, 1H, J=2.6, 5.0 Hz). MS (+, 30 V) calcd for C₇H₈NO₅ [M+H]⁺186.040, found 186.095.

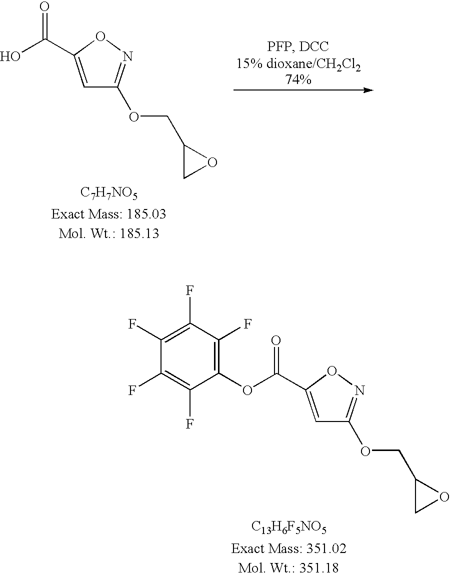

### Core 6

To Core 6 carboxylic acid (673 mg, 3.63 mmol) in 15% dioxane/DCM (45 mL) was added a solution of PFP (810 mg, 4.35 mmol) and 1M DCC (4.35 mL, 4,35 mmol) in DCM (5 mL). The reaction mixture was stirred for 2 hours, cooled for 30 minutes in the refrigerator, filtered and concentrated. Purification by column chromatography (SiO₂, 20% ethyl acetate /hexanes) afforded a white solid (940 mg. 74%).

¹H NMR (300 MHz, CDCl₃) δ6.86 (s, 1H) 4.68 (dd, 1H, J=2.8, 11.8 Hz), 4.22 (dd, 1H, J=6.4, 11.8 Hz), 3.40 (m, 1H), 2.94 (dd, 1H, J=0.7, 4.8 Hz), 2.76 (dd, 1H, J=2.7, 4.9 Hz). MS (+, 30 V) calcd for C₁₃H₆F₅NO₅ [M +H]⁺352.017, found 352.063

### Example 7

### Synthesis of (S)-Core 6

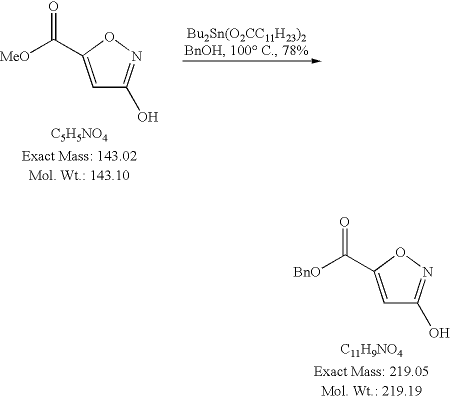

### Benzyl 3-hydroxy-5-isoxazole Carboxylate

Methyl 3-hydroxy-5-isoxazole carboxylate (8.28 g, 57.8 mmol) was heated with benzyl alcohol (30 mL) and dibutyltin dilaurate (1.94 mL, 3.3 mmol, 25 wt %) at 100° C. for 18 hours, according to the procedure described by Leon et al. (J. Am. Chem. Soc. 1996, 118, 8847-8859). The benzyl alcohol was removed in vacuo to afford a rusty brown solid (12.68 g). The solid was dissolved in DCM (100 mL) and washed with water (2×100 mL) and brine (2×100 mL). The organics were dried over sodium sulfate and concentrated. Recrystallization from 30% ethyl acetate/hexane afforded a pale tan solid (9.92 g, 78%).

¹H NMR (300 MHz, CDCl₃) δ7.40 (m, 5H), 6.60 (s, 1H) 5.38 (s, 2H). MS calcd for C₁₁H₁₀NO₄ [M+H]⁺220.057, found 220.058.

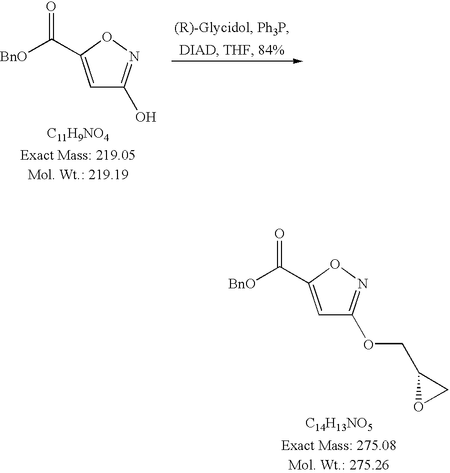

### (S)-Core 6 Benzyl Ester

To benzyl 3-hydroxy-5-isoxazole carboxylate (0.75 g, 3.42 mmol) in THF (20 mL) was added triphenylphosphine (1.1 g, 4.1 mmol), (R)-glycidol (0.3 g, 4.1 mmol) and diisopropyl azodicarboxylate (DIAD) (0.81 mL, 4.1 mmol). The reaction was stirred for 3 hours and concentrated. Purification by column chromatography (SiO₂, 0.5% MeOH/DCM) afforded a slightly impure yellow oil (0.94 g). Recrystallization from 10% ethyl acetate/hexane yielded a white solid (0.79 g, 84%).

¹H NMR (300 MHz, CDCl₃) δ7.40 (m, 5H), 6.59 (s, 1H) 5.37 (s, 2H), 4.60 (dd, 1H, J=2.8, 11.8 Hz), 4.16 (dd, 1H, J=6.3, 11.8 Hz), 3.37 (m, 1H), 2.90 (apparent t, 1H, J=4.6 Hz), 2.73 (dd, 1H, J=2.5, 4.7 Hz). MS calcd for C₁₄H₁₄NO₅ [M+H]⁺276.088, found 276.073

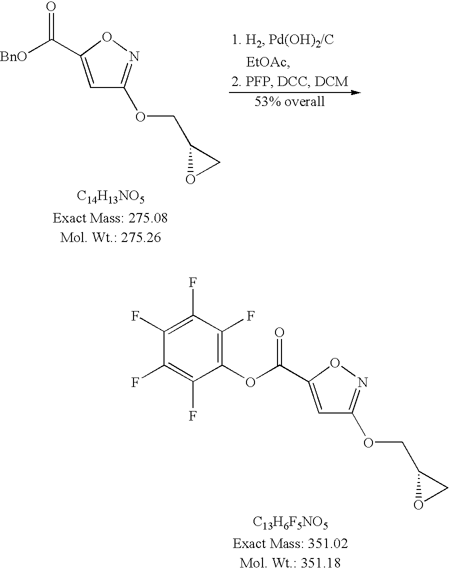

### (S)-Core 6

To (S)-Core 6 benzyl ester (0.73 g, 2.7 mmol) in ethyl acetate (75 mL) under argon was added 20% palladium hydroxide on carbon (75 mg). The reaction mixture was evacuated and flushed with hydrogen (balloon). The reaction was stirred for 1 hour. To the crude reaction mixture was added a solution of PFP (496 mg, 2.7 mmol) and 1M DCC (2.7 mL, 2.7 mmol) in DCM (5 mL). The reaction mixture was stirred for 20 minutes, filtered through Celite and concentrated. Purification by column chromatography (SiO₂, 20% ethyl acetate/hexanes) afforded a white solid (500 mg. 53%).

¹H NMR (300 MHz, CDCl₃) δ6.86 (s, 1H) 4.68 (dd, 1H, J=2.8, 11.8 Hz), 4.22 (dd, 1H, J=6.4, 11.8 Hz), 3.40 (m, 1H), 2.94 (dd, 1H, J=0.7, 4.8 Hz), 2.76 (dd, 1H, J=2.7, 4.9 Hz). MS calcd for C₁₃H₇F₆NO₅ [M+H]⁺352.027, found 352.091.

### Example 8

### Synthesis of (R)-Core 6

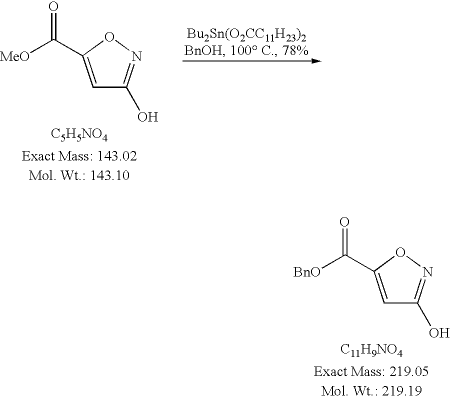

### Benzyl 3-hydroxy-5-isoxazole Carboxylate

Methyl 3-hydroxy-5-isoxazole carboxylate (8.28 g, 57.8 mmol) was heated with benzyl alcohol (30 mL) and dibutyltin dilaurate (1.94 mL, 3.3 mmol, 25 wt %) at 100° C. for 18 hours, according to the procedure described by Leon et al. (J. Am. Chem. Soc. 1996, 118, 8847-8859). The benzyl alcohol was removed in vacuo to afford a rusty brown solid (12.68 g). The solid was dissolved in DCM (100 mL) and washed with water (2×100 mL) and brine (2×100 mL). The organics were dried over sodium sulfate and concentrated. Recyrstallization from 30% ethyl acetate/hexane afforded a pale tan solid (9.92 g, 78%).

¹H NMR (300 MHz, CDCl₃) δ7.40 (m, 5H), 6.60 (s, 1H) 5.38 (s, 2H). MS calcd for C₁₁H₁₀NO₄ [M+H]⁺220.057, found 220.058.

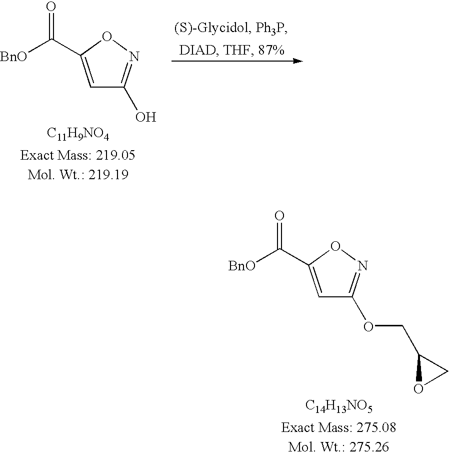

### (R)-Core 6 Benzyl Ester

To benzyl 3-hydroxy-5-isoxazole carboxylate (0.75 g, 3.42 mmol) in THF (20 mL) was added triphenylphosphine (1.1 g, 4.1 mmol), (S)-glycidol (0.3 g, 4.1 mmol) and DIAD (0.81 mL, 4.1 mmol). The reaction was stirred for 2.75 hours and concentrated. Purification by column chromatography (SiO₂, 0.5% MeOH/DCM) afforded a slightly impure yellow oil (0.94 g). Recrystallization from 10% ethyl acetate/hexane yielded a white solid (0.82 g, 87%).

¹H NMR (300 MHz, CDCl₃) δ7.40 (m, 5H), 6.59 (s, 1H) 5.37 (s, 2H), 4.60 (dd, 1H, J=2.8, 11.8 Hz), 4.16 (dd, 1H, J=6.3, 11.8 Hz), 3.37 (m, 1H), 2.90 (apparent t, 1H, J=4.6 Hz), 2.73 (dd, 1H, J=2.5, 4.7 Hz). MS calcd for C₁₄H₁₄NO₅ [M+H]⁺276.088, found 276.072.

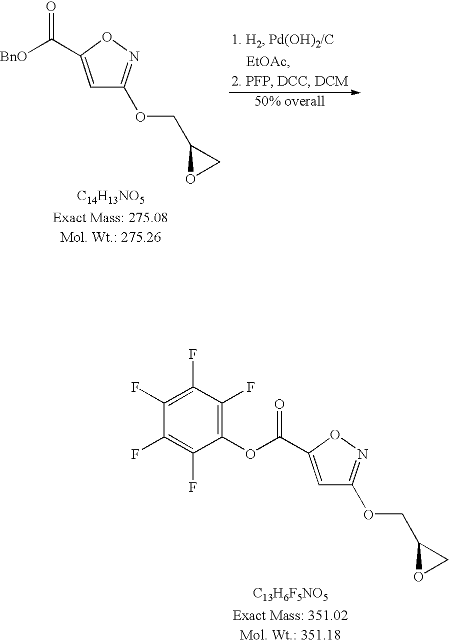

### (R)-Core 6

To (R)-Core 6 benzyl ester (0.82 g, 2.97 mmol) in ethyl acetate (80 mL) under argon was added 20% palladium hydroxide on carbon (100 mg). The reaction mixture was evacuated and flushed with hydrogen (balloon). The reaction was stirred for 30 minutes. To the crude reaction mixture was added a solution of PFP (552 mg, 3.0 mmol) and 1M DCC (3.0 mL, 3.0 mmol) in DCM (5 mL). The reaction mixture was stirred for 20 minutes, filtered through Celite and concentrated. Purification by column chromatography (SiO₂, 20% ethyl acetate/hexanes) afforded a white solid (517 mg. 50%).

¹H NMR (300 MHz, CDCl₃) δ6.86 (s, 1H) 4.68 (dd, 1H, J=2.8, 11.8 Hz), 4.22 (dd, 1H, J=6.4, 11.8 Hz), 3.40 (m, 1H), 2.94 (dd, 1H, J=0.7, 4.8 Hz), 2.76 (dd, 1H, J=2.7, 4.9 Hz). MS calcd for C₁₃H₇F₆NO₅ [M+H]⁺352.027, found 352.091.

### Example 9

### Synthesis of Core 7

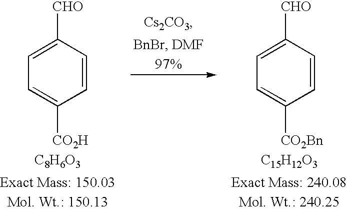

### Benzyl 4-formylbenzoate

To 4-carboxybenzaldehyde (1.50 g, 10 mmol) in DMF (20 mL) was added cesium carbonate (3.58 g, 11 mmol) and benzyl bromide (1.2 mL, 10 mmol). The reaction mixture was stirred for 1.5 hours, filtered and concentrated. The crude mixture was dissolved in 25% ethyl acetate/hexanes and washed with water and brine. The organic layer was dried over sodium sulfate and concentrated to afford a pale yellow solid (2.32 g, 97%).

¹H NMR (300 MHz, CDCl₃) δ10.10 (s, 1H), 8.23 (d, 2H, J=8.3 Hz), 7.95 (d, 2H, J=8.3 Hz), 7.43 (m, 5H), 5.40 (s, 2H).

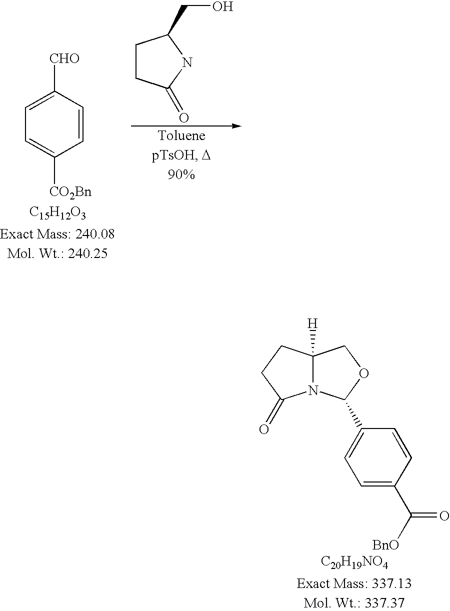

### Bicyclic Lactam Benzyl Ester

A mixture of (S)-(+)-5-(hydroxymethy)-2-pyrrolidinone (1.15 g, 10 mmol), benzyl 4-formybenzoate (3.12 g, 13 mmol) and p-toluenesulfonic acid (p-TsOH) (19 mg, 0.1 mmol) in toluene was heated to reflux with a Dean-Stark trap for 2 hours. After cooling the reaction mixture was diluted with ethyl acetate (50 mL) and washed with saturated sodium bicarbonate solution and brine. The organic layer was dried over sodium sulfate and concentrated. Purification by column chromatography (SiO₂, 1% MeOH/DCM to 2% MeOH/DCM) afforded an orange solid (3.02 g, 90%).

¹H NMR (300 MHz, CDCl₃) δ8.07 (d, 2H, J=8.3 Hz), 7.52 (d, 2H, J=8.3 Hz), 7.41 (m, 5H), 6.36 (s, 1H), 5.37 (s, 2H), 4.23 (dd, 1H, J=6.4, 7.5 Hz), 4.11 (m, 1H), 3.50 (t, 1H, J=8.0 Hz), 2.80 (m, 1H), 2.56 (m, 1H), 1.95 (m, 1H).

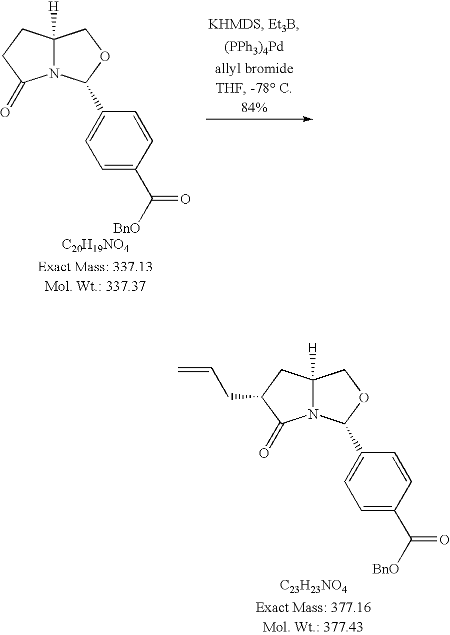

### Allyl Bicyclic Lactam Benzyl Ester

Reference: Zhang, R.; Brownewell, F.; Madalengoitia J. S.; Tet. Let 1999 40, 2707

To bicyclic lactam (3.01 g, 8.94 mmol) in THF (50 mL) at −78° C. was added 0.5 M potassium hexamethyldisilazide (KHMDS) in toluene (20 mL, 10 mmol). The resulting olive colored, cloudy solution was stirred for 15-20 minutes at −78° C. To the reaction mixture was added 1.0 M triethylborane in THF (13.4 mL, 13.4 mmol) at −78° C. The reaction mixture cleared and was stirred for 15 minutes. To the mixture was added tetrakistriphenylphosphine palladium (520 mg, 5 mol %) in THF (10 mL) and allyl bromide (2.32 mL, 26.8 mmol). The reaction mixture was stirred at −78° C. for 1 hour, warmed to room temperature and stirred for an additional 5 hours. It was poured into brine solution and separated. The aqueous layer was extracted with ethyl acetate. The combined organics were dried over sodium sulfate and concentrated. Purification by column chromatography (SiO₂, 3% MeOH/DCM) afforded the product (2.83 g, 84%) as predominately one diastereomer.

¹H NMR (300 MHz, CDCl₃) δ8.06 (d, 2H, J=8.4 Hz), 7.52 (d, 2H, J=8.2 Hz), 7.41 (m, 5H), 6.35 (s, 1H), 5.78 (m, 1H), 5.37 (s, 2H), 5.14 (dd, 1H, J=1.4, 13.4 Hz), 5.13 (dd, 1H, J=1.4, 15.5 Hz), 4.22 (dd, 1H, J=6.3, 8.0 Hz), 4.00 (m, 1H), 3.42 (t, 1H, J=8.0 Hz), 2.78 (m, 1H), 2.56 (m, 1H), 2.39 (m, 1H), 2.12 (d, 1H, J=6.0 Hz), 2.09 (dd, 1H, J=1.2, 4.0 Hz). MS (+, 30 V) calcd for C₂₃H₂₃NO₄ [M+H]⁺378.163, found 378.170

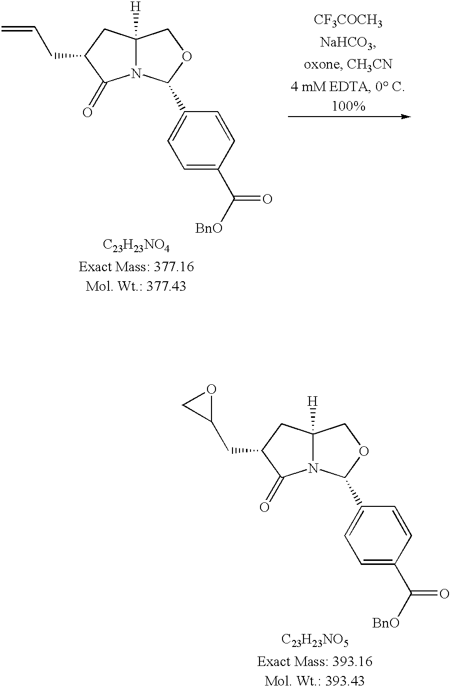

### Core 7 Benzyl Ester

To the bicyclic lactam (1.02 g, 2.70 mmol) in 1:1 acetonitrile:4.0 M NaEDTA solution (50 mL) at 0° C. was added 1,1,1-trifluoroacetone (100 μL). A mixture of sodium bicarbonate (3.70 g, 44 mmol) and Oxone® (5.81 g, 9.45 mmol) were added portionwise. The reaction mixture was allowed to slowly warm to room temperature. Analysis by TLC (3% MeOH/DCM) showed the reaction to be incomplete. The mixture was cooled in an ice bath and addition 1,1,1-trifluoroacetone (200 μL), sodium bicarbonate (3.70 g, 44 mmol) and Oxone® (5.81 g, 9.45 mmol) were added to drive the reaction to completion. The crude mixture was filtered and the solids were washed with DCM. The filtrate was poured into brine and separated. The organic layer was dried over sodium sulfate and concentrated to afford the product (1.06 g, 100%) as a mixture of diastereomers.

¹H NMR (300 MHz, CDCl₃) δ8.07 (d, 2H, J=8.3 Hz), 7.52 (d, 2H, J=8.2 Hz), 7.41 (m, 5H), 6.35 (s, 1H), 5.37 (s, 2H), 4.22 (m, 1H), 4.05 (m, 1H), 3.44 (m, 1H), 3.05 (m, 1H), 2.88 (m, 1H), 2.79 (m, 1H), 2.54 (m, 1H), 2.48 (m, 1H), 2.20 (m, 2H), 1.98 (m, 1H); MS calcd for C₂₃H₂₄NO₅ [M+H]⁺394.167, found 394.130.

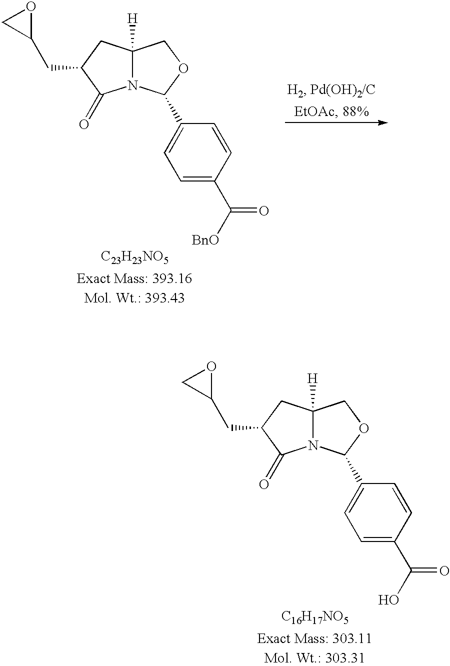

### Core 7 Carboxylic Acid

To Core 7 benzyl ester (2.22 g, 5.64 mmol) in ethyl acetate (50 mL) under argon was added 20 wt % palladium hydroxide on carbon (222 mg). The mixture was evacuated, flushed with hydrogen (balloon) and stirred for 1.5 hours. The mixture was filtered through Celite and concentrated to afford a white foam (1.52 g, 88%).

¹H NMR (300 MHz, CDCl₃) δ8.08 (d, 2H, J=8.1 Hz), 7.54 (d, 2H, J=8.1 Hz), 6,36 (s, 1H) 4.25 (m, 1H), 4.11 (m, 1H), 3.46 (m, 1H), 3.04 (m, 1H), 2.92 (m, 1H), 2.80 (apparent t, 1H, J=4.4 Hz), 2.53 (ddd, 1H, J=2.6, 4.9. 16.3 Hz), 2.26 (m, 2H), 1.96 (m, 1H); MS calcd for C₁₆H₁₈NO₅ [M+H]⁺304.117, found 304.226.

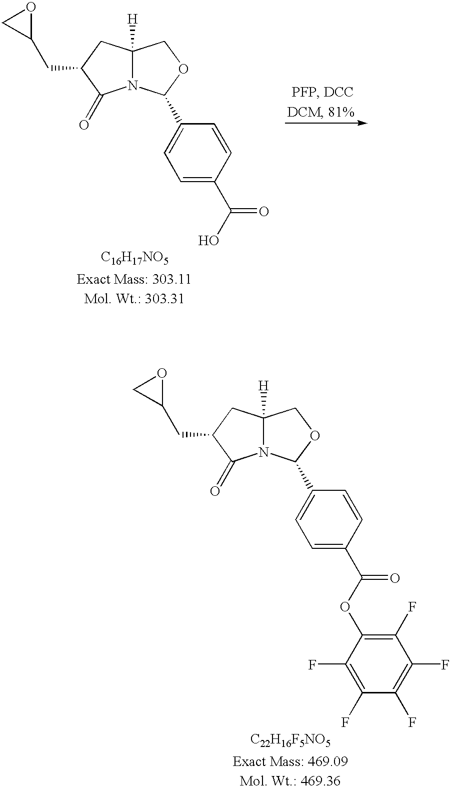

### Core 7

To Core 7 carboxylic acid (1.52 g, 5 mmol) in DCM (40 mL) was added a solution of PFP (1.10 g, 6 mmol) and 1M DCC (6 mL, 6 mmol) in DCM (5 mL). The reaction mixture was stirred for 2 hours, cooled and filtered. Purification by column chromatography (SiO₂, 50% ethyl acetate/hexanes) afforded a white solid (1.89 g, 81%) as a mixture of diastereomers.

¹H NMR (300 MHz, CDCl₃) (major isomer listed first) δ8.19 (d, 2H, J=8.3 Hz), 7.65 (d, 2H, J=8.2 Hz), 6.39 and 6,38 (s, 1H) 4.29 (apparent t, 1H, J=7.8 Hz), 4.08 (m, 1H), 3.47 and 3.48 (apparent t, 1H, J=8.5 Hz), 3.05 (m, 1H), 2.89 (m, 1H), 2.80 apparent t, 1H, J=6.5 Hz), 2.55 and 2.53 (dd, 1H, J=2.6, 4.9. Hz), 2.27 (m, 2H), 1.96 (m, 1H) MS (+, 30 V) calcd for C₂₂H₁₆F₅NO₅ [M+H]⁺470.095, found 470.130

### Example 10

### Synthesis of Core 8

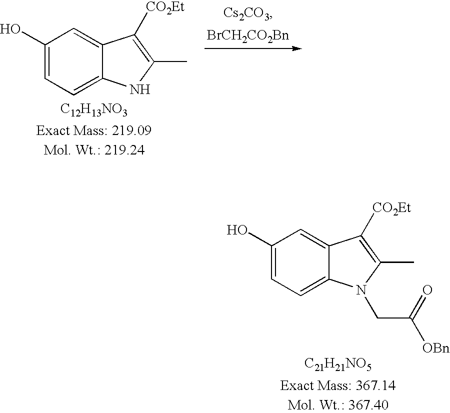

### Core 8 Benzyl Ester Phenol

Ethyl 5-hydroxy-2-methylindole-3-carboxylate was purchased from Aldrich Chemical Company (E3,180-4) and was used as received. To a solution of ethyl 5-hydroxy-2-methylindole-3-carboxylate (4.0 g, 18.2 mmol) in THF (80 mL) and DCM (240 mL) was added cesium carbonate (pulverized, 11.9 g, 36.5 mmol) and benzyl bromoacetate (3.2 mL, 20.1 mmol). A solvent ratio of 1:3 THF:DCM was found to improve the chemoselectivity of N-alkylation over O-alkylation.

The reaction was stirred for 24 hours at room temperature. Monitoring of the a reaction by TLC (5% MeOH/DCM) revealed it to be incomplete. Cesium carbonate was added (9.5 g, 1.6 equiv), and the reaction was stirred for 8 hours before more cesium carbonate (4.8 g, 0.8 equiv) and benzyl bromoacetate (0.46 mL, 2.9 mmol) were added. After 24 hours, a final addition of benzyl bromoacetate (0.93 mL, 0.3 equiv) and stirring for another 24 hours was necessary to drive the reaction to completion. The suspension was filtered, concentrated, and cooled in an ice bath to afford a solid. This was dissolved in 1% MeOH/DCM and chromatographed (SiO₂, 1-5% MeOH/DCM) to give the desired compound (1.09 g). Another pure portion of material could be obtained from the crude chromatography fractions by concentration and precipitation with DCM:toluene (1:10) to give a combined total of 3.8 g (57%) of purified product.

¹H NMR (300 MHz, CDCl3) δ8.21 (br s, 1H), 7.58 (d, 1H, J=2.6 Hz), 7.30 (m, 5H), 7.15 (d, 1H, J=8.8 Hz), 6.87 (dd, 1H, J=2.5, 8.7 Hz), 5.23 (s, 2H), 4.73 (s, 2H), 4.33 (q, 2H, J=7.1 Hz), 2.70 (s, 3H), 1.40 (t, 3H, J=7.1 Hz). MS calcd for C₂₁H₂₂NO₅ [M+H]⁺368.147, found 368.121.

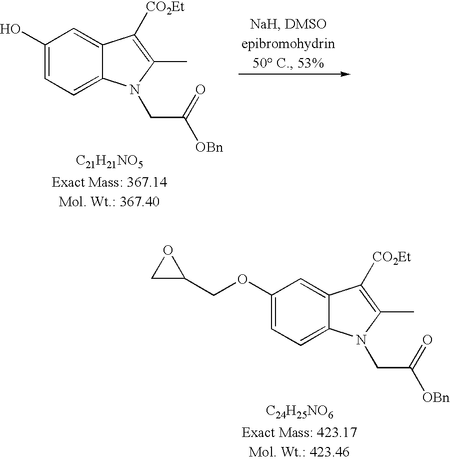

### Core 8 Benzyl Ester

To indole (587 mg, 1.60 mmol) in DMSO was added 60% sodium hydride (82 mg, 2.05 mmol). The reaction mixture was stirred for 1 hour. To the mixture was added epibromohydrin (0.205 mL, 2.4 mmol). The mixture was heated to 50° C. and stirred overnight. After cooling the mixture was poured into ice water and extracted with 50% ether/ethyl acetate. The combined organic layers were washed with water and brine, dried over sodium sulfate and concentrated. Purification by column chromatography (SiO₂, 1% MeOH/DCM) afforded a pale yellow oil (356 mg, 53%).

¹H NMR (300 MHz, CDCl3) δ7.63 (d, 1H, J=2.5 Hz), 7.37 (d, 1H, J=4.4 Hz), 7.33 (m, 5H), 7.22 (d, 1H, J=8. Hz), 6.94 (dd, 1H, J=2.6, 8.9 Hz), 5.25 (s, 2H), 4.75 (s, 2H), 4.46 (dd, 1H, J=2.9, 15.8 Hz), 4.35 (q, 2H, J=7.1 Hz), 4.17 (dd, 1H, J=4.8, 15.8 Hz), 3.21 (m, 1H), 2.76 (m, 4H), 2.41 (dd, 1H, J=2.5, 4.6 Hz), 1.40 (t, 3H, J=7.1 Hz).

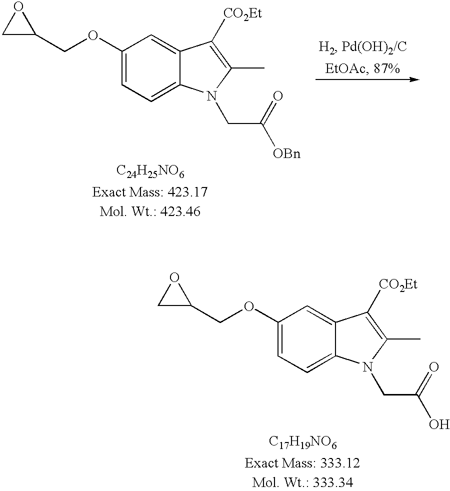

### Core 8 Carboxylic Acid

To Core 8 benzyl ester (356 mg, 0.84 mmol) in ethyl acetate (15 mL) was added 20% palladium hydroxide on carbon (40 mg) and the reaction was evacuated and back filled with a hydrogen balloon. After 30 minutes the reaction was complete and filtered through Celite using ethyl acetate to give a white solid (244 mg, 87%).

¹H NMR (300 MHz, CDCl3) δ7.66 (d, 1H, J=2.5 Hz), 7.23 (s, 1H), 6.94 (dd, 1H, J=2.6, 8.8 Hz), 4.74 (s, 2H), 4.48 (dd, 1H, J=2.8, 15.7 Hz), 4.38 (q, 2H, J=7.1 Hz), 4.13 (dd, 1H, J=2.4, 4.8 Hz), 3.23 (m, 1H), 2.78 (apparent s, 4H), 2.41 (m, 1H).

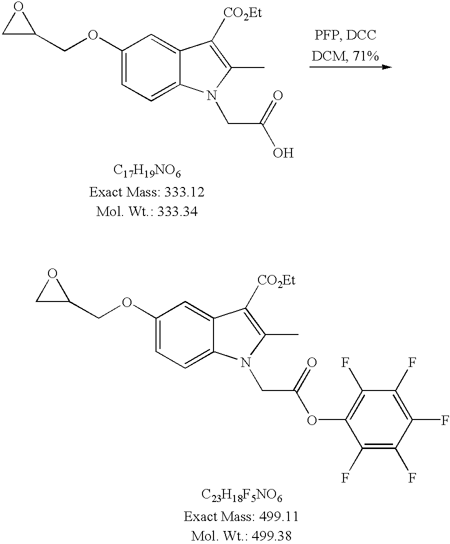

### Core 8

To Core 8 carboxylic acid (244 mg, 0.73 mmol) in DCM (10 mL) was added a solution of PFP (162 mg, 0.88 mmol) and 1M DCC (0.9 mL) in DCM (2 mL). After 30 minutes the reaction was complete and was cooled in an ice bath, filtered, and concentrated. Column chromatography (SiO₂, 25% hexanes/DCM) afforded a white solid (258 mg, 71%).

¹H NMR (300 MHz, CDCl₃) δ7.73 (d, 1H, J=2.6 Hz), 7.27 (d, 1H, J=4.4 Hz), 6.98 (dd, 1H, J=2.4, 8.7 Hz), 5.07 (s, 2 H), 4.50 (dd, 1H, J=2.6, 15.7 Hz), 4.40 (q, 2H, J=7.1 Hz), 4.20 (dd, 1H, J=4.8, 15.7 Hz), 3.25 (m, 1H), 2.79 (appar. t, 1H, J=4.6 Hz), 2.78 (s, 3H), 2.42 (dd, 1H, J=2.4, 4.6 Hz), 1.44 (t, 3H, J=7.0 Hz).

MS (+, 30 V) calcd for C23H19F5NO6 [M+H]+500.113, found 500.177.

### Example 11

### Synthesis of Core 9

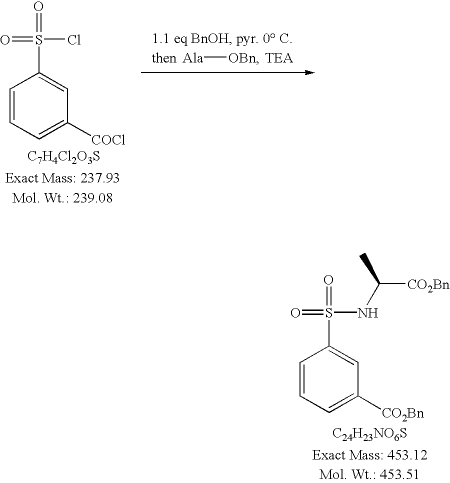

### Core 9 Benzyl Ester Sulfonamide

3-(Chlorosulfonyl)benzoyl chloride was purchased from Acros Chemical Company and used as received. To 3-(chlorosulfonyl)benzoyl chloride (20.2 g, 84.4 mmol) in DCM (600 mL) at 0° C. was added benzyl alcohol (9.6 mL, 93 mmol) and pyridine (dropwise, 8.2 mL, 101 mmol). After 20 minutes at 0° C. the ice bath was removed and the reaction allowed to warm to room temperature over 1 hour under a calcium chloride drying tube at which time DMAP (40 mg, cat) was added. TLC analysis (60:40 hexane:ethyl acetate) confirmed the esterification reaction to be complete. Alanine benzyl ester hydrochloride (20 g, 93 mmol) and triethylamine (36.5 mL, 262 mmol) were added and the reaction was stirred at room temperature for 3 hours, diluted with DCM, and rinsed with 1N HCl, saturated aqueous bicarbonate, dried over sodium sulfate, and concentrated to clear oil (30.2 g, 79%), which required no further purification.

¹H NMR (300 MHz, CDCl₃) δ8.51 (br s, 1H), 8.22 (d, 1H, J=7.8 Hz), 7.99 (d, 1H, J=7.7 Hz), 7.54-7.16 (m, 11H), 5.38 (s, 2H), 5.28 (br d, 1H, J=9 Hz), 4.93 (s, 2H), 4.06 (m, 1H), 1.40 (d, 3H, J=7.2 Hz).

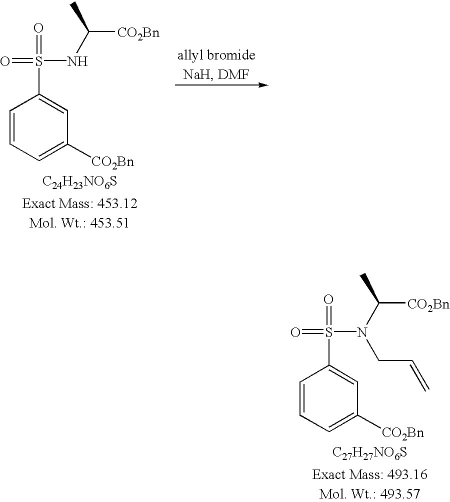

### Core 9 Benzyl Ester (allyl) Sulfonamide

To a chilled solution of the secondary sulfonamide (42.3 g, 93.4 mmol) in DMF (215 mL) was added sodium hydride (60% dispersion in mineral oil, 5.22 g, 130 mmol) and the mixture stirred for 1 hour, warming slowly to room temperature. Allyl bromide (11.3 mL, 130 mmol) was added and the reaction was stirred for 16 hours under a drying tube at which time TLC analysis (70:30 hexanes:ethyl acetate) indicated that the reaction was complete. The reaction mixture was concentrated, diluted with ethyl acetate, rinsed with water (3 times), and dried over sodium sulfate. The crude reaction mixture was chromatographed (SiO₂, 70:30 hexanes:ethyl acetate) to give the desired compound as a solid (13.0 g, 28%).

¹H NMR (300 MHz, CDCl₃) δ8.50 (br s, 1H), 8.18 (d, 1H, J=7.8 Hz), 7.96 (d, 1H, J=7.7 Hz), 7.47-7.19 (m, 12H), 5.73 (m, 1H), 5.14 (d, 1H, J=17.1 Hz), 5.03 (d, 1H, J=10 Hz), 4.94 (d, 2H, J=2.6 Hz), 4.72 (q, 2H, J=2.5 Hz), 3.94 (dd, 1H, J=5.9, 16 Hz), 3.78 (dd, 1H, J=6.1, 16 Hz), 1.43 (d, 3H, J=7.3 Hz).

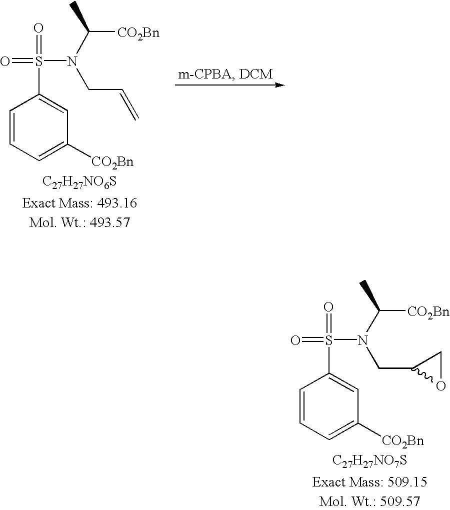

### Core 9 Benzyl Ester (epoxide) Sulfonamide

To the allyl compound (12.7 g, 25.7 mmol) in DCM (140 mL) was added m-chloroperbenzoic acid (m-CPBA) (70% purity grade, 9.5 g, 38.6 mmol) and the reaction mixture was warmed to reflux for 20 hours. Another addition of m-CPBA (4.75 g, 19.2 mmol) was made and the reaction refluxed for another 20 hours before it was diluted with DCM, rinsed with bicarbonate, brine, dried over sodium sulfate, and concentrated to afford an oil (11.6 g, 89%), as a mixture of two diastereomers). Chromatography (SiO₂, 75:25 hexanes:ethyl acetate) was used to purify the product(s) from the starting materials. A small quantity of the less polar diastereomer could be isolated in pure form and is characterized below. However, the bulk of the material contained both diastereomers.

Less polar diastereomer:

¹H NMR (300 MHz, CDCl₃) δ8.47 (t, 1H, J=1.6 Hz), 8.18 (d, 1H, J=7.8 Hz), 7.93 (d, 1H, J=7.8 Hz), 7.93-7.12 (m, 11H), 5.38 (s, 2H), 4.87 (AB quartet, 2H, J=6.5 Hz), 4.76 (q, 1H, J=7.3 Hz), 3.68 (dd, J=3.4, 16 Hz), 3.16 (m, 1H), 2.98 (dd, 1H, J=3.5, 6.5), 2.76 (apparent t, 1H, J=4.4 Hz), 2.50 (dd, 1H, J=2.6, 4.7 Hz), 1.55 (d, 3H, J=7.3 Hz).

MS (both diastereomers) calcd for C27H28NO7S [M+H]⁺510.159, found 510.144.

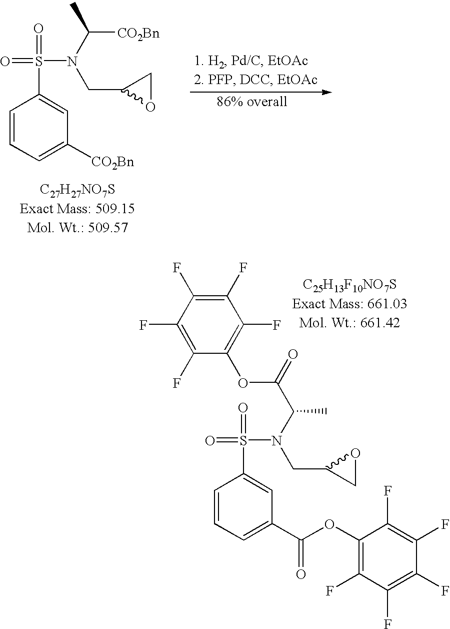

### Core 9

Note: The diacid is unstable and should be used immediately to prevent cyclization onto the epoxide. In this optimized procedure, it is activated in situ to provide the tricore.

To dibenzyl ester epoxide (mixture of both diastereomers, 4.04 g, 7.93 mmol) in ethyl acetate (75 mL) under argon was added Pd/C (10 wt %, 400 mg) and the system was twice evacuated and pressurized with a hydrogen balloon. After 1 hour, TLC (30% ethyl acetate in hexanes) confirmed that the reaction was complete. To this mixture was added a premixed solution of PFP (3.06 g, 16.7 mmol) and DCC (1 M in DCM, 16.7 mL, 16.7 mmol) and the reaction was stirred for 20 minutes at which time the reaction was judged complete by TLC (20% ethyl acetate in hexanes). Filtration through Celite followed by concentration afforded crude Core 9 which was purified using column chromatography (SiO₂, 20% ethyl acetate in hexanes) to afford three groups of fractions. The first crop contained the less polar diastereomer (2.22 g, 42%), the second was a mixture of the two diastereomers (0.71 g, 14%), and the third was the more polar diastereomer (1.58 g, 30%).

Less polar diastereomer:

¹H NMR (300 MHz, CDCl₃) δ8.65 (t, 1H, J=1.7 Hz), 8.38 (dd, 1H, J=1.1, 7.8 Hz), 8.17 (dd, 1H, J=1.2, 7.7 Hz), 7.72 (t, 1H, J=7.9 Hz), 5.18 (q, 1H, J=7.4 Hz), 3.93 (dd, 1H, J=2.8, 16 Hz), 3.31 (m, 1H), 2.93 (dd, 1H, J=6.9, 16 Hz), 2.89 (apparent t, 1H, J=4.4 Hz), 2.61 (dd, 1H, J=2.7, 4.5 Hz), 1.80 (d, 3H, 7.5 Hz).

More polar diastereomer:

¹H NMR (300 MHz, CDCl₃) δ8.68 (t, 1H, J=1.6 Hz), 8.40 (dd, 1H, J=1.2, 7.8 Hz), 8.21 (dd, 1H, J=1.4, 7.7 Hz), 7.71 (t, 1H, J=7.9 Hz), 5.16 (q, 1H, J=7.4 Hz), 3.64 (dd, 1H, J=3.9, 16 Hz), 3.40 (dd, 1H, J=5.8, 16 Hz), 3.20 (m, 1H), 2.83 (apparent t, 1H, J=4.3 Hz), 2.61 (dd, 1H, J=2.7, 4.8 Hz), 1.71 (d, 3H, 7.4 Hz).

MS calcd for C25H14F10NO7S [M+H]⁺662.033, found 662.032.

### Example 12

### Synthesis of Core 10

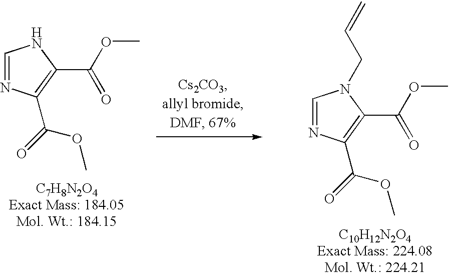

### N-Allylimidazole Dimethyl Ester

Dimethyl 4,5-imidazoledicarboxylate is commercially available. However, for this experiment it was prepared from the diacid using the thionyl chloride /MeOH method. The compound is insoluble in acetone.

To dimethyl 4,5-imidazoledicarboxylate (500 mg, 2.72 mmol) in DMF (10 mL) was added cesium carbonate (0.98 g, 3.0 mmol) and allyl bromide (0.35 mL, 4.1 mmol) and the reaction was stirred overnight. Filtration and concentration yielded a pure solid (502 mg, 82%).

¹H NMR (300 MHz, CDCl₃) δ7.54 (s, 1H), 5.95 (m, 1H), 5.28 (dd, 1H, J=1.0, 10.5 Hz), 5.13 (d, 1H, J=17.0 Hz), 4.82 (dd, 2H, J=1.4, 7.0 Hz), 3.92 (s, 3H), 3.89 (s, 3H).

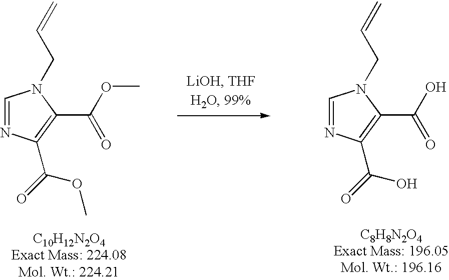

### N-Allylimidazole Dicarboxylic Acid

To the N-allylimidazole dimethyl ester (500 mg, 2.23 mmol) in THF (20 mL) was added water (4 mL) and 1N LiOH (6.5 mL, 6.5 mmol). After 3 hours, amberlite IR-120 resin was added to the cloudy reaction mixture until pH=2-3, and the mixture was filtered and concentrated. To the resultant solid was added toluene, and the mixture evaporated to give the desired compound (407 mg, 93%).

¹H NMR (300 MHz, DMSO) δ8.79 (s, 1H), 6.04 (m, 1H), 5.21 (dd, 1H, J=1.1, 10.3 Hz), 5.11 (m, 3H).

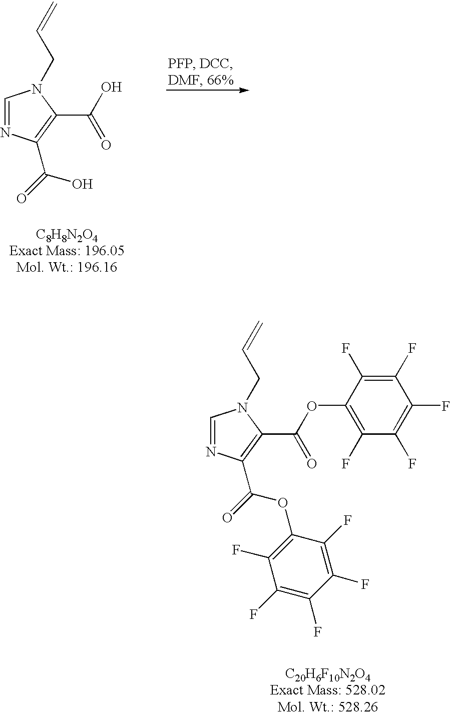

### N-Allylimidazole di(pentafluorophenyl) Ester

To the N-allylimidazole diacid (405 mg, 2.06 mmol) in DMF (10 mL) was added a W5 premixed solution of PFP (836 mg, 4.54 mmol) and IM DCC in DCM (4.5 mL, 4.5 mmol). The reaction was complete (TLC, DCM) after 1 hour. The reaction mixture was filtered, concentrated, and purified using column chromatography (SiO₂, DCM) to give a white solid (615 mg, 57%). Further purification can be performed by recrystallization from 30% ethyl acetate in hexanes.

¹H NMR (300 MHz, CDCl₃) δ7.84 (s, 1H), 6.00 (m, 1H), 5.40 (d, 1H, J=10.3 Hz), 5.25 (d, 1H, J=17.0 Hz), 4.96 (d, 2H, J=5.7 Hz). MS calcd for C₂₀H₇F₆N20₄ [M+H]⁺529.027, found 528.976.

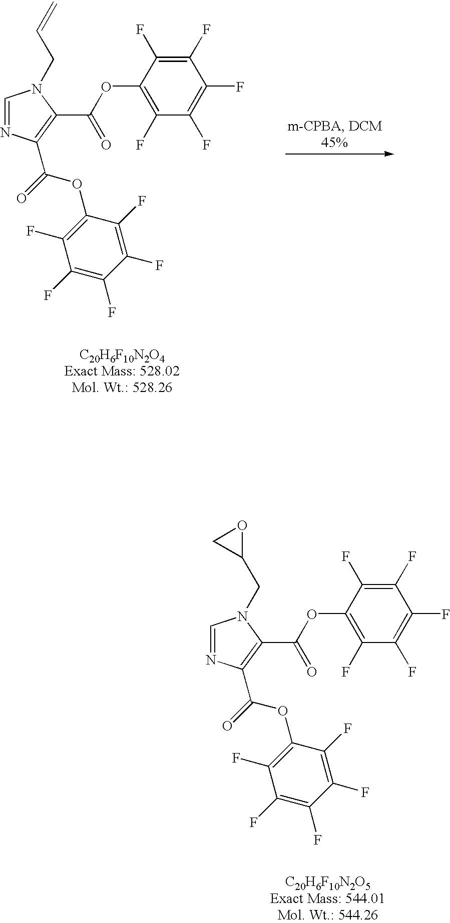

### Core 10

To the N-allyl di(pentafluorophenyl) ester (2.49 g, 4.7 mmol) in DCM (30 mL) was added m-CPBA (90% purity, 1.80 g, 9.4 mmol), and the reaction mixture was warmed to reflux for 72 hours. The reaction was cooled to room temperature, filtered, diluted with DCM, rinsed with aqueous sodium sulfite, bicarbonate, brine, and the organic layer dried over sodium sulfate. Column chromatography (SiO₂, 5% ethyl acetate in DCM) afforded a yellow glassy solid (1.15 g, 45%).

¹H NMR (300 MHz, CDCl₃) δ7.89 (s, 1H), 4.84 (dd, 1H, J=2.4, 15 Hz), 4.23 (dd, 1H, J=6, 15 Hz), 3.38 (m, 1H), 2.95 (apparent t, 1H, J=4.1 Hz), 2.56 (dd, 1H, J=2.5, 4.3 Hz). MS (+, 30 V) calcd for C₂₀H₇F₁₀N₂O₅ [M+H]+545.020, found 544.953.

### Example 13

### Synthesis of Core 11

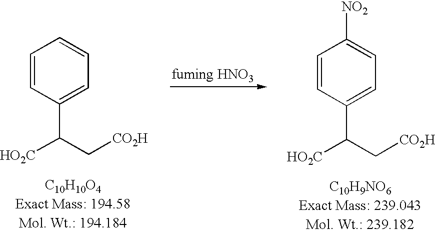

### 4-Nitrophenylsuccinic Acid

Racemic phenylsuccinic acid was purchased from Aldrich and used as received. To fuming nitric acid (25 mL) at 0° C. was added phenylsuccinic acid (5.0 g, 26 mmol) portionwise, and the resultant orange solution was stirred for 10 minutes at 0-4° C. before ice was added. The resultant off-white precipitate was collected via filtration and twice recrystallized from water (200 mL) to give the nitro compound as a pale yellow solid (2.22 g, 36%).

¹H NMR (300 MHz, DMSO) δ12.5 (br s, 2 H), 8.19 (d, 2H, J=8.6 Hz), 7.60 (d, 2H, J=8.7 Hz), 4.11 (dd, 1H, J=5.6, 9.5 Hz), 3.01 (dd, 2H, J=9.6, 17 Hz), 2.65 (dd, 2H, J=5.7, 17 Hz).

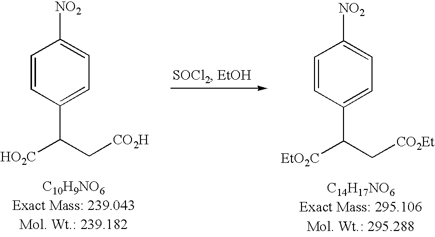

### Diethyl 2-(4-nitrophenyl)succinate

In the following reaction, the 4-nitrophenylsuccinic acid prepared as described above was used. If commercial 4-nitrophenylsuccinic acid is used, the purity should be checked by NMR prior to use.

To 4-nitrophenylsuccinic acid (5.0 g, 21 mmol) in ethanol (100 mL) was slowly added thionyl chloride (10 mL), and the mixture was stirred for 1 hour at room temperature. After warming to reflux for 3 hours, the reaction mixture was cooled to ambient temperature and concentrated. The yellow residue was taken up in DCM (100 mL), rinsed with 1N KOH (50 mL) and dried over sodium sulfate. Concentration afforded an orange oil (5.6 g, 100%).

¹H NMR (300 MHz, CDCl₃) δ8.19 (d, 2H, J=8.6 Hz), 7.47 (d, 2H, J=8.6 Hz), 4.18 (m, 5H), 3.19 (dd, 2H, J=5, 17 Hz), 2.71 (dd, 2H, J=6.1, 17 Hz), 1.23 (t, 3H, J=7.1 Hz), 1.21 (t, 3H, J=7.1 Hz).

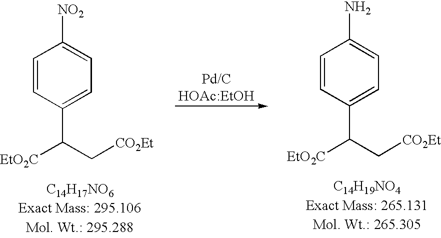

### Diethyl 2-(4-aminophenyl)succinate

To diethyl 2-(4-nitrophenyl)succinate (500 mg, 1.87 mmol) in 1:1 acetic acid:ethanol (20 mL) was added Pd/C (50 mg, 10 wt %), and the flask was evacuated and back-filled twice with a hydrogen balloon. After 48 hours, TLC (60/40 hexanes/ethyl acetate) indicated that the reaction had reached completion. The mixture was filtered through Celite and concentrated. The dark oil was dissolved in DCM (30 mL), rinsed with 1N NaOH, and dried over sodium sulfate. The red-brown oil (427 mg, 96%) required no further purification.

¹H NMR (300 MHz, CDCl₃) δ7.05 (d, 2H, J=8.4 Hz), 6.59 (d, 2H, J=8.4 Hz), 4.25-4.05 (m, 4H), 3.93 (dd, 1H, J=5.5, 10 Hz), 3.12 (dd, 2H, J=5.3, 17 Hz), 2.60 (dd, 2H, J=5.4, 17 Hz), 1.23 (t, 3 H, J=7 Hz), 1.21 (t, 3H, 7 Hz).

MS (+, 15 V) calcd for C14H20NO4 [M+H]⁺266.139, found 266.092

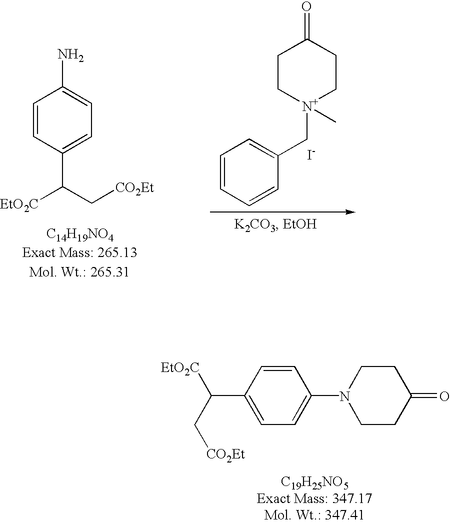

### Core 11 Piperidone Diethyl Ester

The piperidone insertion procedure can be found in: Tortolani, D. R.; Poss, M. A. Org. Lett. 1999, 1, 1261-1262. Preparation of the piperidone salt is also given in this reference.

To aminosuccinic acid (11.2 g, 42.2 mmol) in ethanol (100 mL) was added potassium carbonate (0.82 g, 5.9 mmol) and the reaction was warmed to reflux. The flask was then elevated out of the oil bath and the piperidone salt (21 g, 63.3 mmol) was added along with water (30 mL) in alternate portionwise additions, all whilst the reaction vessel was still hot. The reaction was then placed back in the hot oil bath, bringing the reaction back to reflux. After 3 hours the reaction mixture was cooled to room temperature, concentrated to a red oil, and diluted with DCM (250 mL) and water (100 mL). The DCM layer was isolated, dried over sodium sulfate, and concentrated to a red-brown oil. Column chromatography (SiO₂, 0.5 to 2% MeOH in DCM) afforded an orange oil (10.6 g, 72%).

¹H NMR (300 MHz, CDCl₃) δ7.21 (d, 2H, J=8.6 Hz), 6.90 (d, 2H, J=8.6 Hz), 4.18-4.06 (m, 4H), 4.00 (dd, J=5.5, 10 Hz), 3.59 (apparent t, 4H, J=6.0 Hz), 3.15 (dd, 1H, J=10, 17 Hz), 2.63 (dd, 1H, J=5.5, 17 Hz), 2.54 (apparent t, 4H, J=6.0 Hz), 1.23 (t, 3H, J=7.1 Hz), 1.21 (t, 3H, J=7.1 Hz).

MS (+, 30V) calcd for C19H26NO5 [M+H]⁺348.181, found 348.199.

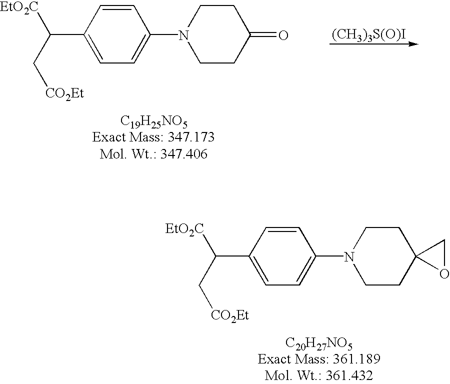

### Core 11 Diethyl Ester Epoxide

To trimethylsulfoxonium iodide (7.05 g, 32.0 mmol) in anhydrous DMSO (50 mL) at room temperature was added sodium hydride (60% dispersion in mineral oil, 1.34 g, 33.6 mmol), and the reaction mixture was warmed to 40° C. After 1.5 hours, the piperidone (10.6 g, 30.5 mmol) in anhydrous DMSO (20 mL) was added via addition funnel. During the course of the reaction, the color was observed to change from blue-green to black to light brown. After 2.5 hours the reaction was cooled to room temperature and was poured onto ice water (150 mL), extracted with ether (6×150 mL), rinsed with brine, and dried over sodium sulfate to afford the product (9.0 g, 80%), which required no further purification.

¹H NMR (300 MHz, CDCl₃) δ7.17 (d, 2H, J=8.6 Hz), 6.90 (d, 2H, J=8.6 Hz), 4.05 (m, 4H), 3.96 (dd, 1H, J=5.4, 10 Hz), 3.43-3.32 (m, 4H), 3.16 (dd, 1H, J=10, 17 Hz), 2.71 (s, 2H), 2.62 (dd, J=5.3, 17 Hz), 2.00-1.96 (m, 2H), 1.65-1.61 (m, 2H), 1.22 (t, 3H, J=7.0 Hz), 1.20 (t, 3H, J=7.0 Hz).

MS calcd (+, 30 V) for C20H28NO5 [M+H]⁺362.197, found 362.088.

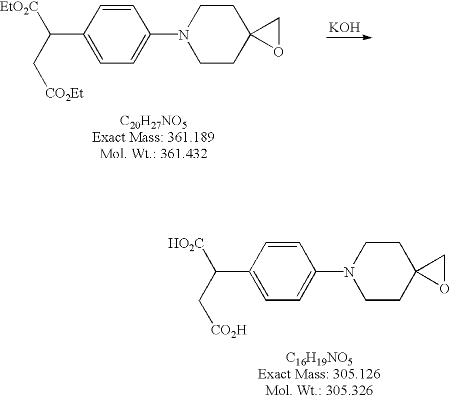

### Core 11 Diacid Epoxide

To the diethyl ester (80 mg, 0.23 mmol) in THF (5 mL) and water (1 mL) at 0° C. was added 1N KOH (0.5 mL, 0.5 mmol), and the reaction mixture was allowed to warm to room temperature overnight. TLC (5% MeOH/DCM) revealed that no ester-containing compounds remained. The organics were evaporated, the pH was set to 3-4 with 1N HCl, the aqueous layer was extracted with ethyl acetate (6×20 mL), and the organic layer was dried over sodium sulfate and concentrated to a beige solid (66 mg, 94%).

¹H NMR (300 MHz, DMSO) δ12.3 (br s, 2H), 7.11 (d, 2H, J=8.5 Hz), 6.92 (d, 2H, J=8.6 Hz), 3.77 (dd, 1H, J=4.9, 10 Hz), 3.3 (m, 4H), 2.87 (dd, 1H, J=10, 17 Hz), 2.66 (s, 2H), 2.46 (dd, 1H, J=5, 17 Hz), 1.80 (m, 2H), 1.57 (m, 2H).

MS (−, 30 V) calcd for C16H18NO5 [M−H]⁻304.118, found 304.102.

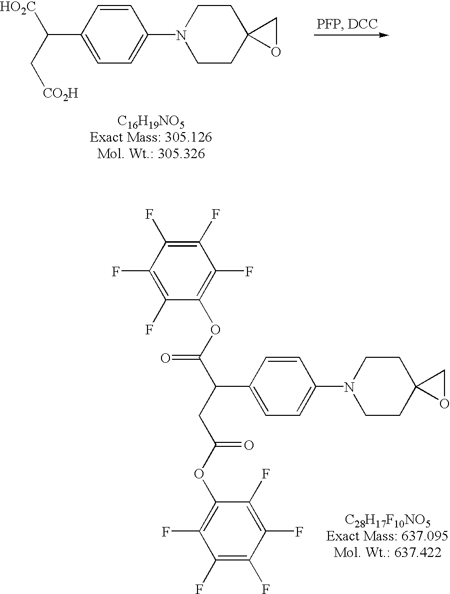

### Core 11

To the diacid (66 mg, 0.22 mmol) in dioxane (10 mL) was added a premixed solution of DCC (0.5 mL, 0.47 mmol), PFP (88 mg, 0.47 mmol), and DCM (2 mL). After a 4-hour reaction period, the reaction mixture was cooled in an ice bath, filtered, and concentrated. The crude material was semi-purified using column chromatography (SiO₂, 10% to 25% ethyl acetate in DCM) to afford an orange oil (43 mg, 29%) which contained DCC-based impurities. Column chromatography (SiO₂) proved to be an undesirable method of purification due to the reactivity of the product with silica gel.

The product, after several treatments with cold dioxane followed by filtration of undesired insolubles, could only be purified to ˜80% purity.

¹H NMR (300 MHz, CDCl₃) δ7.27 (d, 2H, J=8.7 Hz), 6.97 (d, 2H, J=8.7 Hz), 4.45 (dd, 1H, J=5.4, 10 Hz), 3.60 (dd, 1H, J=10, 17 Hz), 3.53-3.33 (m, 4H), 3.13 (dd, 1H, 5.4, 17 Hz), 2.73 (s, 2H), 2.04-1.91 (m, 2H), 1.7-1.63 (m, 2H).

### Example 14

### Synthesis of Core 12

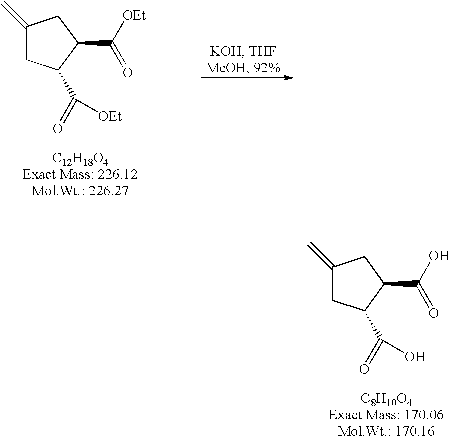

### Core 12 Alkene Diacid

trans-Diethyl-4-methylene-1,2-cyclopentanedicarboxylate was purchased from Lancaster Chemical Company (16215) or E-Merck Chemical Company (8.14188.0010), and was used as received.

To the alkene diester (31.2 g, 138 mmol) in THF (500 mL) and MeOH (200 mL) was added 2N KOH (275 mL, 551 mmol) portionwise at 0° C. The bright yellow reaction was allowed to warm to room temperature overnight. The organics were removed in vacuo, the remaining basic solution cooled in an ice bath and acidified to pH 3-4 using 1N HCl (note: it is suggested that the pH be set instead to 1-2 and that 6M HCl be used to reduce the volume). Ethyl acetate was added and the layers separated and the organic layer dried over sodium sulfate to give a solid (13.4 g, 58%).

¹H NMR (300 MHz, DMSO) δ12.4 (br s, 2H), 4.89 (m, 2H), 3.31-2.94 (m, 2H), 2.63 (dd, 2H, J=4.7, 16 Hz), 2.44-2.36 (m, 21H).

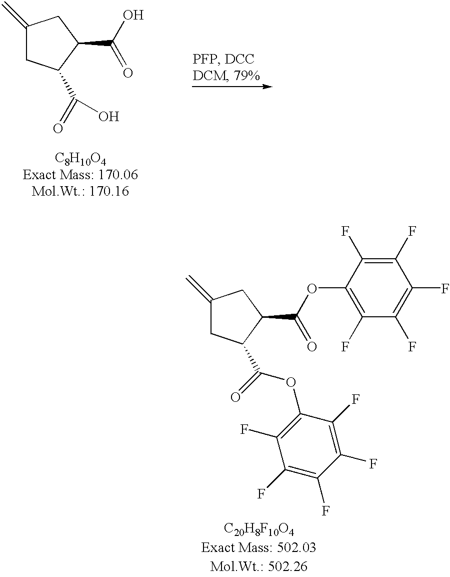

### Core 12 Alkene di(pentafluorophenyl) Ester

To the alkene diacid (13.4 g, 78.7 mmol) was added DCM (400 mL) and a premixed solution of DCC (1M in DCM, 173 mL, 173 mmol) and PFP (31.8 g, 173 mmol). After 70 minutes the reaction was cooled to −20° C. for 1 hour, filtered, and concentrated to give an orange solid. Column chromatography (SiO₂, DCM) provided the pure product as a white solid (33.3 g, 79%).

¹H NMR (300 MHz, CDCl₃) δ5.07 (s, 2H), 3.68 (m, 2H), 3.04 (dd, 2H, J=6.5, 15 Hz), 2.84 (dd, J=6.5, 15 Hz).

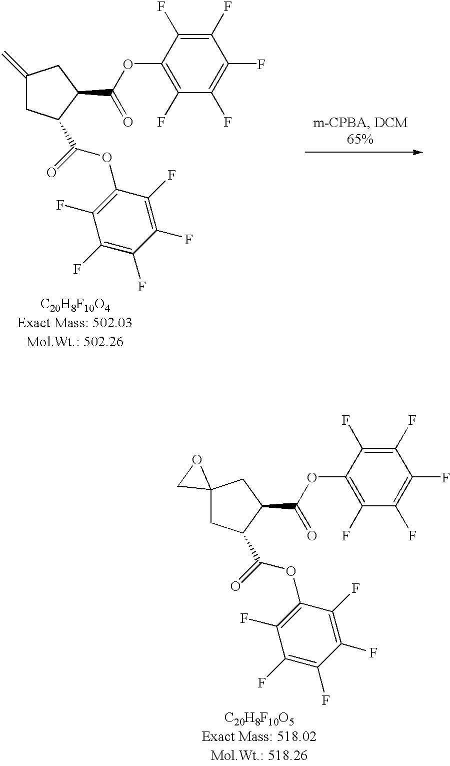

### Core 12

To a chilled (0° C.) solution of the alkene di(pentaflurophenyl) ester (17 g, 32 mmol) in DCM (200 mL) was added M-CPBA (90% purity, 12.2 g, 64 mmol), and the solution was allowed to warm to ambient temperature overnight. The reaction mixture was filtered, concentrated, and recrystallized from hexane/ethyl acetate (175 mL/24 mL) to give white needles in two pure crops (total of 11.5 g, 65%).

¹H NMR (300 MHz, CDCl₃) δ3.90 (q, 1H, J=8.9 Hz), 3.75 (q, 1H, J=9.0 Hz), 2.96 (m, 2H), 2.67 (dd, 1H, J=9.6, 14.7 Hz), 2.54 (dd, 1H, J=9.9, 14.2 Hz), 2.33 (m, 2H); MS calcd for C₂₀H₉F₁₀O₅ [M+H]⁺519.027, found 519.131.

### Example 15

### Preparation of a Combinatorial Library from Core 6

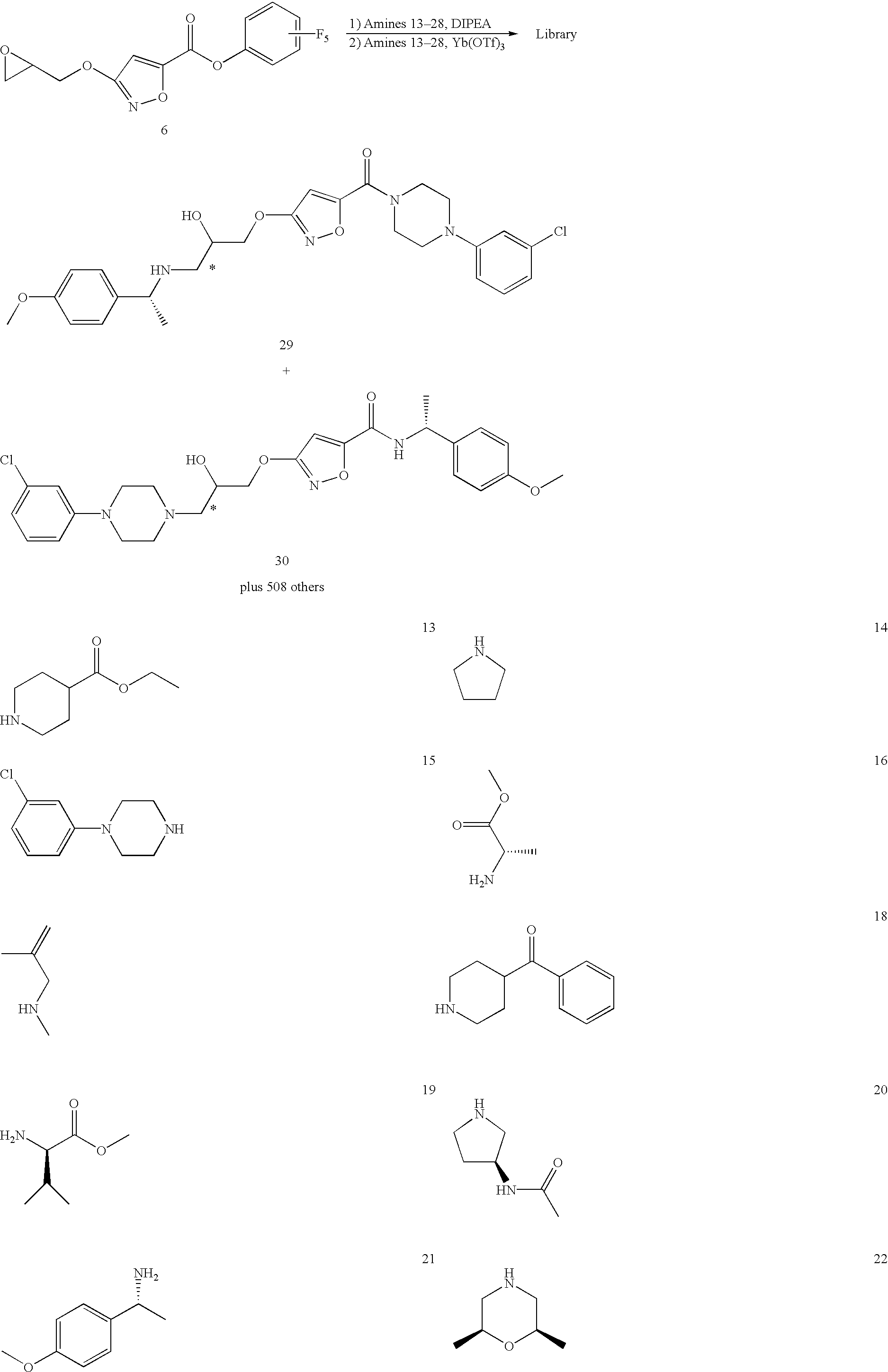

To a solution of Core 6 (100 mg, 0.28 mmol) in DCM/THF (3 mL each) at 24° C. was added a solution of building blocks 13-28 (0.019 mmol each) as a solution in DCM/THF (3 mL each) and an Yb(OTf)₃ catalyst solution (100 μL of a 120 mg solution in 1.5 mL THF) and DIEA (50 μL, 0.28 mmol) were added. The mixture was heated to 45-50° C. for 24 hours, and then cooled. Amberlite (100 mg) was added and the reaction mixture was stirred for an additional 1 hour at 24° C. The reaction mixture was then filtered and concentrated to yield the solution-phase library as a slightly yellow film containing 512 substitutionally and stereochemically unique compounds. LC-MS analysis indicated that 94% of the expected masses were found.

This library was combined with four similar libraries to yield a mixture of 2560 compounds. Pooling libraries allows significantly more compounds to be screened per unit time than does screening in a non-multiplexed fashion. A total of 1500 similar 2500-member libraries were screened, representing >3,500,000 compounds, consuming a total of 3.0 mg of protein.

### Example 16

### Automated Ligand Identification System (ALIS) Screening of E. coli Dihydrofolate Reductase (DHFRec)

### Methods for Handling DHFR Prior to ALIS Screening

E. coli DHFR was purified to apparent homogeneity by SDS-PAGE according to standard procedures [Baccanari, D. P., Stone, D., and L. Kuyper J. Biol. Chem. 256: 1738-1747 (1981)]. The process of DHFR preparation that preceded ALIS screening consisted of the following components: (1) combination of purified DHFR with drug libraries to form a binding mixture; (2) incubation of DHFR-library binding mixture; (3) introduction of the DHFR-library binding mixture into ALIS system. The binding mixture was prepared to yield the following final composition: 50 μM DHFR and 2.5 mM library in aqueous “DHFR Buffer” containing 100 mM NaCl, 0.1 mM EDTA, 50 mM Tris-pH 7.5, 0.1 mM dithioerythritol, and 2.5% dimethyl sulfoxide. This DHFR-library binding mixture was then incubated at ambient temperature for thirty minutes to permit protein-ligand interactions to reach equilibrium. Inoculated samples were then stored at four degrees Celsius until was introduced into the ALIS screening system. Upon introduction into ALIS screening, the SEC buffer composition was identical to “DHFR Buffer.” Subsequent manipulations are described under the descriptions of ALIS methods.

### Complex Formation

To 1 μL of a DMSO solution of 100 mM 2500-member library was added 32.3 μL pre-warmed (37° C.), pH 7.5, 50 mM phosphate buffer containing 100 mM NaCl and 0.1 mM dithioerythritol. The resulting solution was mixed by repeated pipetting and centrifuged at 10,000 g for 10 minutes. A 10 μL aliquot of the supernatant was added to 2 μL of a 60 μM solution of purified DHFR in pH 7.5, 50 mM phosphate buffer containing 100 mM NaCl and 0.1 mM dithioerythritol, and mixed by repeated pipetting. The sample was incubated at room temperature for 30 minutes and then was chilled at 4° C. pending affinity selection-mass spectrometric analysis. The final sample thus contained a cumulative library compound concentration of 2.5 mM (at 0.8 μM/component) with 10 μM DHFRec in a final volume of 10 μL 50 mM pH 7.5 phosphate buffer containing 100 mM NaCl and 2.5% DMSO. As such, 8 pmol of each library component and 100 pmol (1.8 μg) of protein were utilized in a single analysis. By using excess protein relative to each library member, competition between multiple binders in a given library is precluded.

### Affinity Selection-Mass Spectrometry

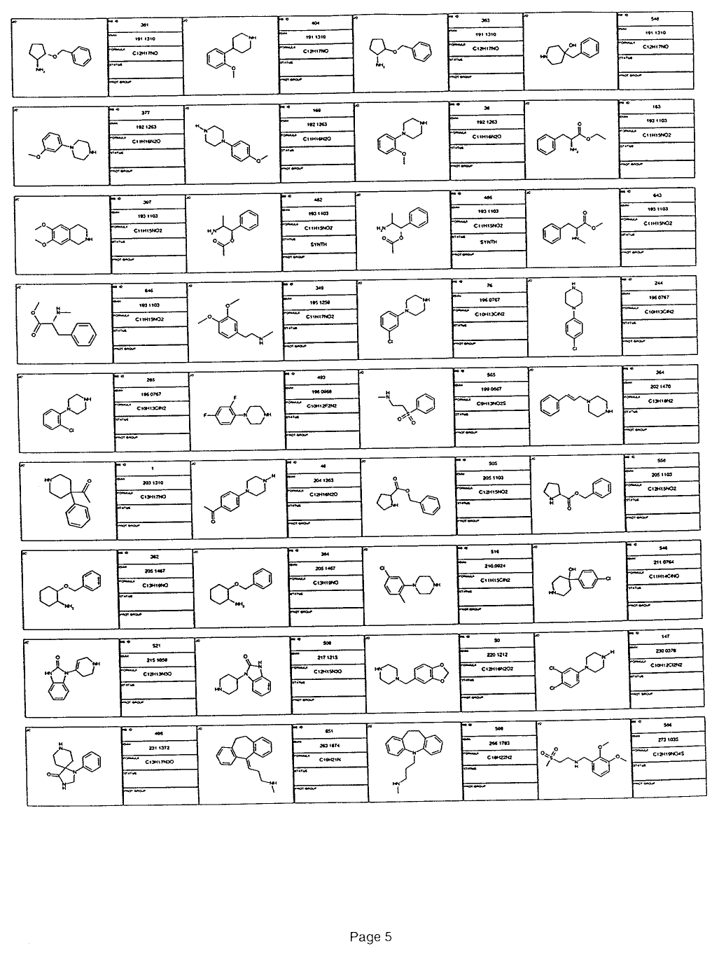

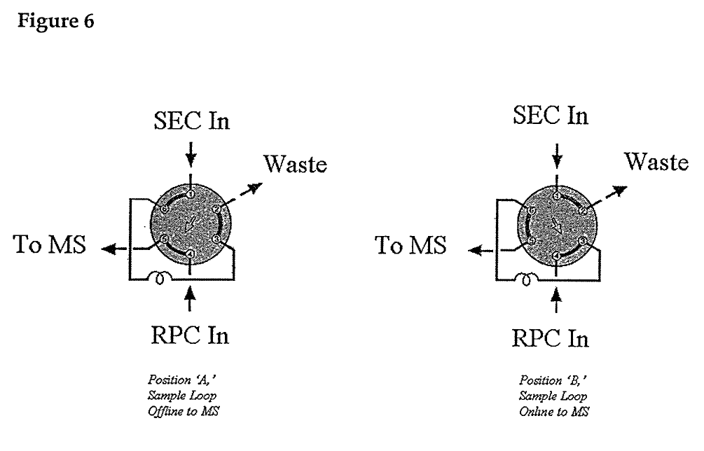

Size exclusion chromatography was performed over 4.6 mm×50 mm×5 μm SEC columns (Poly-LC Inc., polyhydroxyethyl A 60 Å) at 0° C. using 50 mM pH 7.5 phosphate buffer containing 100 mM NaCl and 2.5% DMSO at a flow rate of 2 mL/minute. The SEC band containing the complex was identified using a UV-VIS detector monitoring 230 nm and transferred by way of a sample loop to a low-flow (100 μL/min) reverse-phase chromatography (RPC) system (FIG. 6). The RPC column (Higgins C-18 (1 mm×50 mm×5 μm particulate); Higgins Analytical) was maintained at 60° C. to promote dissociation of ligands from the complex. The ligand was eluted into a high-resolution mass spectrometer for analysis using a gradient of 5% to 95% acetonitrile (0.1% formic acid) in water (0.1% formic acid) over five minutes.

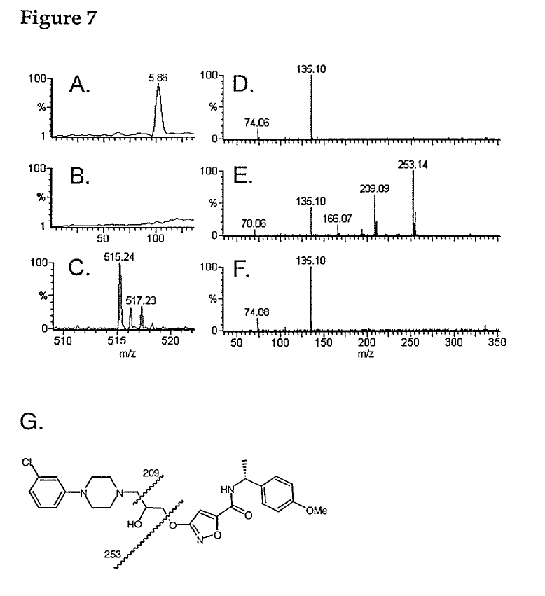

ALIS screening of the library of Example 15, multiplexed as described above, yielded a monochlorinated ligand of molecular weight 514.23 amu. Two of the five combined libraries contained monochlorinated members within 0.05 amu of this molecular weight; independent affinity selection experiments with these two libraries confirmed that the ligand originated from the library of Example 15 (FIGS. 7A, B, and C). Correlation of the found mass with the calculated masses of the library components identified the ligand as either 29 or 30.

### Example 17

### MS-MS Identification of a DHFRec Ligand

Affinity selection-MS-MS experiments were conducted to determine the regioisomeric connectivity of the building blocks in the ligand. A smaller library was prepared as described above, except that building blocks 15 and 21 alone were used in combination with Core 6. The positional isomers 29 and 30 in this sublibrary were readily separated by reverse-phase chromatography (same conditions as described above, except that a gradient of 20 minutes was used; T_{R}=15.1, 15.7 minutes) for structural assignment by LC-MS-MS. A spectrum of the ligand obtained in an affinity selection LC-MS-MS experiment with the sublibrary matched that assigned to regioisomer 29 (FIGS. 7A, B, and C). Isomer 30 yielded diagnostic ions for its assignment (FIG. 7G).

### Example 18

### Synthesis and Characterization of Discrete Compounds

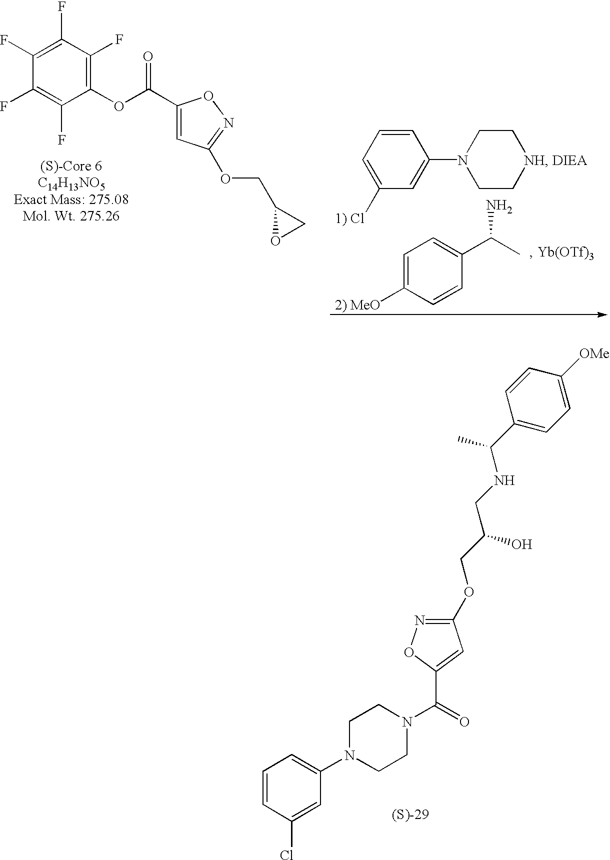

To (S)-Core 6 (100 mg, 0.28 mmol) in DCM/THF (3 mL each) at 0° C. was added 1-(3-chlorophenyl)-piperazine (59 mg, 0.30 mmol) and DIEA (98 μL, 0.56 mmol). The reaction was allowed to warm to room temperature over 2 h and was diluted with DCM. The reaction was then transferred to a separatory funnel and rinsed with 1N citric acid and water. The organic layer was dried over sodium sulfate and concentrated to give a clear oil. To the intermediate dissolved in DCM/iPrOH (3 mL:1 mL) was added (R)-1-(4-methoxyphenyl)ethylamine (50 μL, 0.40 mol), Yb(OTf)₃ catalyst solution (100 μL of a 120 mg solution in 1.5 mL THF), and DIEA (50 μL, 0.28 mmol). The mixture was heated to 45-50° C. for 24 h before it was concentrated and chromatographed (SiO₂, 1-5% MeOH/DCM) to give (S)-29 as a clear oil, which was freeze dried to a hygroscopic powder (81 mg, 56%).

¹H NMR (300 MHz, CDCl₃) δ7.40 (m, 5H), 6.59 (s, 1H) 5.37 (s, 2H), 4.60 (dd, 1H, J=2.8, 11.8 Hz), 4.16 (dd, 1H, J=6.3, 11.8 Hz), 3.37 (m, 1H), 2.90 (app. t, 1H, J=4.6 Hz), 2.73 (dd, 1H, J=2.5, 4.7 Hz). MS calcd for C₁₄H₁₄NO₅ [M+H]⁺276.088, found 276.073

### Example 19

### Competitive ALIS Experiments

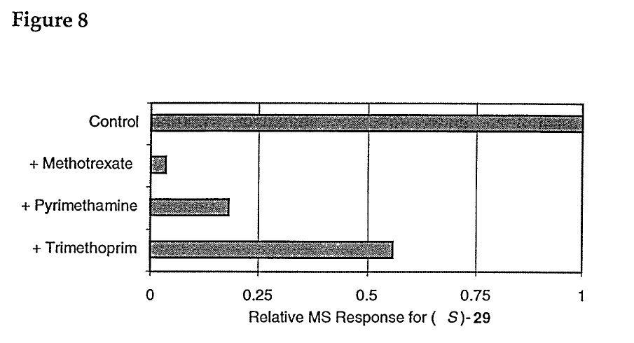

The antibiotics methotrexate, pyrimethamine and trimethoprim are well-characterized DHFRec inhibitors (McCormack, in Comprehensive Medicinal Chemistry, Hansch et al., Eds., Pergamon Press: Elmsford, N.Y., 1990, Vol. 2, pp 271-298). Tiara et al, J. Med. Chem., 31: 129-137 (1998) reports that methotrexate has a K_{d} value of 0.96 nM. Pattishall et al., J. Biol. Chem., 251: 7011-7020 (1976), reports that pyrimethamine has a K_{d} value of 47 nM and trimethoprim has a K_{d} value of 1.4 μM. Competitive ALIS experiments with (S)-29 and these drugs were conducted by combining 15 μM DHFRec with 80 μM (S)-29 in the absence (control) and presence of 80 μM of each drug (FIG. 8). Competitive binding was observed for all three drugs, with the magnitude of the suppression of binding by (S)-29 correlating with the affinity of each competitor.

### Example 20

### Determination of Antibacterial Activity

Protocols for the measurement of IC₅₀ for the evaluation of antibacterial activity followed standard methods [Antibiotics in Laboratory Medicine, V. Lorian, Ed. Fourth edition, 1996. Williams and Wilkins; Baltimore, Md.]. Briefly, purified (S)-29, antibiotic control, or vehicle (DMSO) was subjected to serial dilution into Luria-Bertani growth medium in a manner that placed 0.1 mL aliquots of medium-plus-vehicle or medium-plus-antibiotic into each well of a sterile polystyrene 96-well plate. To aid compound dissolution, medium-analyte mixtures were warmed to 37° C. for eight hours with agitation. Bacterial cells, [E. coli: hsdS gal (λcIts857 ind 1 Sam7 nin5 lacUV5-T7 gene 1); BL21(DE3)] were grown in to mid-log phase at 37° C. The culture was then diluted 1:10⁵ into prewarmed growth medium. Aliquots of diluted bacterial culture (0.1 mL) were then combined with the pre-aliquotted medium-analyte mixtures to yield 0.2 mL inoculated cultures containing no more than 2.5% DMSO. The plated inoculate series were then grown for twenty-four hours at 37° C. with orbital shaking. Bacterial cell growth was quantitated by measuring absorbance at 600 nm. IC₅₀ for the inhibition of bacterial growth was defined as the concentration of drug at which cell growth was half maximal at twenty-four hours.

Bioactivity assessments conducted against E. coli indicate that (S)-29 inhibits bacterial growth with a mean IC₅₀ of 56 μM (29 μg/mL) The mean IC₅₀ of the diastereomer (R)-29 (prepared as described in Example 18, but starting with (R)-Core 6) was determined to be 190 μM (98 μg/mL). These IC₅₀ values correlate well with the observed binding affinities. The regioisomeric control compounds (S)-30 and (R)-30 showed no measurable growth inhibition.

### Example 21

### Determination of Specificity for Pathogen vs. Host DHFR

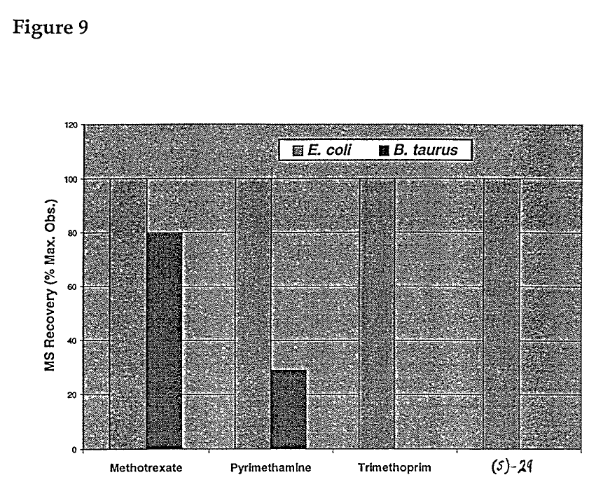

Utilizing the ALIS techniques described above, the affinity of ligands for DHFRec was compared to the affinity of the same ligands for the bovine (B. taurus) DHFR enzyme. As shown in FIG. 9, (S)-29 selectively binds to the E. coli enzyme, while exhibiting no detectable affinity for the B. taurus enzyme. This selectivity profile mirrors that of trimethoprim, a prescribed antibacterial agent. By contrast, methotrexate exhibits affinity for both the E. coli and the B. taurus enzymes. Consequently, methotrexate has significant toxicity in man.

While the foregoing invention has been described in some detail for purposes of clarity and understanding, it will be appreciated by one skilled in the art from a reading of this disclosure that various changes in form and detail can be made without departing from the true scope of the invention and appended claims.

What is claimed is:

1. A method of forming a combinatorial library of compounds, the method comprising reacting a plurality of core molecules with a mixture of nucleophilic building blocks in a reaction vessel to form a library of compounds, wherein each of said core molecules comprises (i) an acid halide, sulfonyl halide, isocyanate or isocyanate equivalent, or activated ester functional group; and (ii) an epoxide functional group.

2. The method of claim, 1, wherein the mixture of nucleophilic building blocks comprises at least one amine.

3. The method of claim 1, wherein substantially all of the nucleophilic building blocks are amines.

4. The method of claim 1, wherein substantially all of the core molecules are the same.

5. The method of claim 1, wherein the plurality of core molecules comprises at least two different core molecules.

6. The method of claim 3, wherein said reacting step comprises sequentially
(i) contacting the core molecules with a mixture of amine building blocks so that reaction with the acid halide or activated ester functional groups is achieved; and
(ii) adding a Lewis acid so that reaction of the amine building blocks with the epoxide functional groups is achieved.

7. The method of claim 4, wherein the core molecule comprises two acid halide, sulfonyl halide, isocyanate or isocyanate equivalent, or activated ester functional groups.

8. The method of claim 4, wherein the core molecule comprises three acid halide, sulfonyl halide, isocyanate or isocyanate equivalent, or activated ester functional groups.

9. The method of claim 1, wherein each of said core molecules comprises an activated ester group.

10. The method of claim 9, wherein the activated ester group is a pentafluorophenyl ester group.

11. The method of claim 9, wherein the activated ester group is a dinitrophenyl ester group.

12. The method of claim 1, wherein the epoxide functional group is a terminal epoxide.

13. The method of claim 1, wherein said core molecule has the formula
A-B-C,
wherein
B comprises from 1 to about 4 carbocyclic or heterocyclic rings, any of which rings may be optionally substituted, and wherein A and C may be attached to the same or different rings;
A is an organic moiety comprising an acid halide, sulfonyl halide, isocyanate or isocyanate equivalent, or activated ester functional group; and
C is an organic moiety comprising an epoxide functional group.

14. The method of claim 13, wherein at least one of the rings is an aromatic ring.

15. The method of claim 13, wherein B comprises a fused bicyclic or tricyclic ring system.

16. The method of claim 13, wherein B comprises two rings connected by a covalent bond.

17. The method of claim 13, wherein A and C are attached to the same ring.

18. The method of claim 14, wherein the ring to which A and C are attached is a benzene ring.

19. The method of claim 13, wherein
A has the formula
—Y¹—W,
where:
W is an isocyanate or isocyanate equivalent, acid halide, or sulfonyl halide functional group, or W has the formula
—C(O)—OR¹,
where R¹ is selected from the group consisting of imido, haloalkyl, and aryl substituted with at least one electron withdrawing substituent;
Y¹ is absent or comprises a linking chain of from 1 to about 6 contiguous atoms independently selected from the group consisting of carbon, nitrogen, oxygen, or sulfur, wherein the carbon and nitrogen atoms may be optionally substituted and the nitrogen and sulfur atoms may be optionally oxidized, and wherein any of the contiguous atoms of the chemical linkage may form part of a ring structure; and
C has the formula
—Y²—Z,
where Z is an epoxide, which may be optionally substituted with an alkyl, aryl, aralkyl, or carboalkoxy group, and Y² is as defined above for Y¹.

20. The method of claim 19, wherein R¹ is selected from the group consisting of succinimide, phthalimide, perfluoroalkyl, pentafluorophenyl, dinitrophenyl, nitrophenyl, difluorophenyl, fluorophenyl, trifluorophenyl, chlorophenyl, dichlorophenyl, chloronitrophenyl, and tetrafluoronitrophenyl.

21. The method of claim 19, wherein X is selected from the group consisting of halo, dinitrophenyloxy and pentafluorophenyloxy.

22. The method of claim 19, wherein Y¹ comprises a —C(O)—X group.

23. The method of claim 19, wherein Y² comprises a —C(O)—X group.

24. The method of claim 19, wherein the Y¹ or Y²linking chain comprises an ester, amide or sulfonamide linkage.

25. The method of claim 19, wherein the Y¹ or Y² linking chain comprises an ether linkage.

26. The method of claim 19, wherein the Y² linking chain comprises a ring and the epoxide functional group is a spiroepoxide attached to the ring.

27. The method of claim 1, wherein said mixture of amines comprises primary amines.

28. The method of claim 1, wherein at least 90% of said library compounds each comprise a β-hydroxyamine functional group and an amide, sulfonamide, or urea functional group.

29. The method of claim 28, wherein at least 95% of said library compounds each comprise a β-hydroxyamine functional group and an amide functional group.

30. The method of claim 29, wherein at least 99% of said library compounds each comprise a β-hydroxyamine functional group and an amide functional group.

31. The method of claim 4, wherein said core molecule has the formula

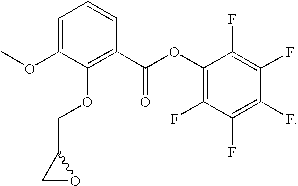

32. The method of claim 4, wherein said core molecule has the formula

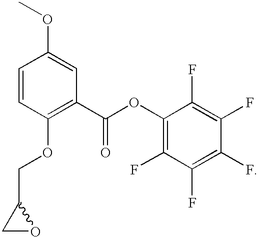

33. The method of claim 4, wherein said core molecule has the formula

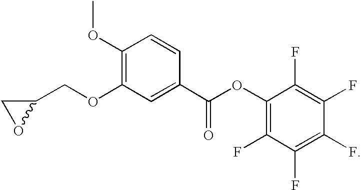

34. The method of claim 4, wherein said core molecule has the formula

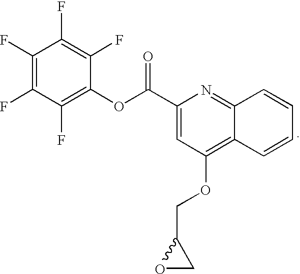

35. The method of claim 4, wherein said core molecule has the formula

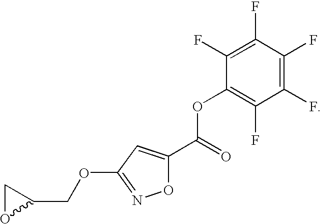

36. The method of claim 4, wherein said core molecule has the formula

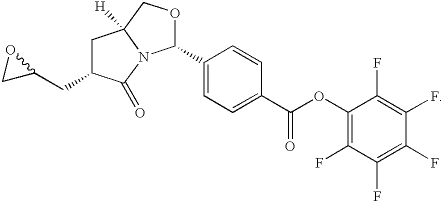

37. The method of claim 4, wherein said core molecule has the formula

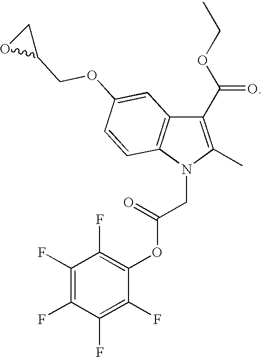

38. The method of claim 4, wherein said core molecule has the formula

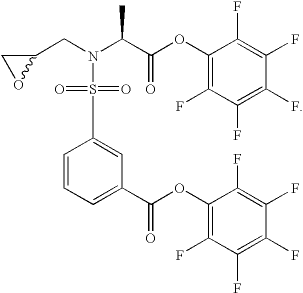

39. The method of claim 4, wherein said core molecule has the formula

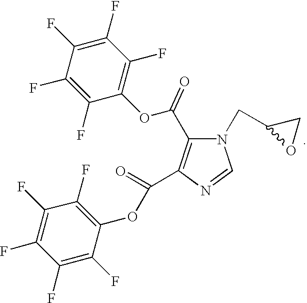

40. The method of claim 4, wherein said core molecule has the formula

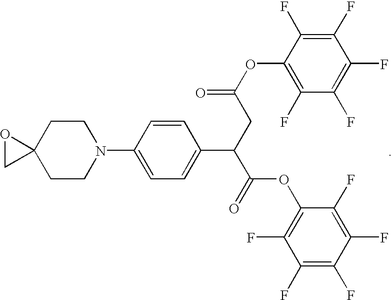

41. The method of claim 4, wherein said core molecule has the formula

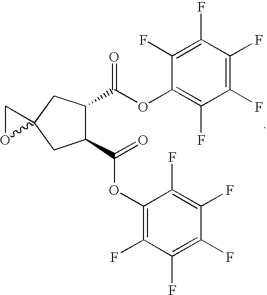

42. A combinatorial library of compounds, wherein each of said compounds is produced from the reaction of a plurality of core molecules with a mixture of nucleophilic building blocks, wherein the core molecule comprises (i) an acid halide, sulfonyl halide, isocyanate or isocyanate equivalent, or activated ester functional group; and (ii) an epoxide functional group.

43. The library of claim 42, wherein said reacting step comprises sequentially
(i) contacting the core molecules with a mixture of amine building blocks so that reaction with the acid halide or activated ester functional groups is achieved; and
(ii) adding a Lewis acid so that reaction of the amine building blocks with the epoxide functional groups is achieved.

44. A compound having the formula
A-B-C,
wherein
B comprises from 1 to about 4 carbocyclic or heterocyclic rings, any of which rings may be optionally substituted, and wherein A and C may be attached to the same or different rings;
A is an organic moiety comprising an acid halide, sulfonyl halide, isocyanate or isocyanate equivalent, or activated ester functional group; and
C is an organic moiety comprising an epoxide functional group.
